(19) United States
(12) Patent Application Publication
(10) Pub. No.: US 2024/0280589 A1
(43) Pub. Date: Aug. 22, 2024

(54) USE OF FIBRONECTIN IN PREDICTING PREECLAMPSIA

(71) Applicants: Sinai Health System, Toronto (CA); The Hospital for Sick Children, Toronto (CA)

(72) Inventors: Isabella Caniggia, Toronto (CA); Martin Post, Toronto (CA); Sruthi Alahari, Mississauga (CA)

(21) Appl. No.: 18/391,104

(22) Filed: Dec. 20, 2023

Related U.S. Application Data
(60) Provisional application No. 63/448,395, filed on Feb. 27, 2023.
(60) Provisional application No. 63/445,210, filed on Feb. 13, 2023.

(51) Int. Cl. G01N 33/68

(52) U.S. Cl. CPC G01N 33/689; G01N 2333/78; G01N 2333/90245; G01N 2800/368

(57) ABSTRACT

A method and kit for predicting preeclampsia is provided. Levels of Jumonji C domain containing protein 6 (JMJD6) and fibronectin are measured in placenta-derived small extracellular vesicles isolated from a subject. The levels of JMJD6 and fibronectin are compared to a non-preeclamptic control. If levels of JMJD6 are depressed and levels of fibronectin are elevated, the subject may be monitored or treated for preeclampsia.

### CROSS-REFERENCE TO RELATED APPLICATION(S)

This application claims the benefit of U.S. Provisional Patent Application No. 63/445,210 entitled “Fibronectin and JMJD6 signature in circulating placental extracellular vesicles for the detection of preeclampsia”, filed Feb. 13, 2023, and U.S. Provisional Patent Application No. 63/448,395 entitled “Fibronectin and JMJD6 signature in circulating placental extracellular vesicles for the detection of preeclampsia”, filed Feb. 27, 2023, the entire contents of which are incorporated herein by reference.

### FIELD

The present specification is directed to methods of predicting preeclampsia, and specifically predicting preeclampsia by measuring the jumonji c domain containing protein 6 enzyme (JMJD6).

### BACKGROUND

Preeclampsia (PE) is a major obstetric complication that accounts for 10% of maternal morbidity and mortality globally. Currently, there are limited tools to assess placental health and function in crucial gestational periods for diagnosis and early intervention. Although excessive fibronectin has been detected in placentae collected after delivery and termination from women with preeclampsia, obtaining placental samples during pregnancy is highly invasive. Furthermore, the accuracy and validity of fibronectin as a biomarker of preeclampsia remains debatable. Therefore, there is a need for a reliable, non-invasive test that can detect both early- and late-onset preeclampsia.

### SUMMARY

In one aspect, the specification provides the use of fibronectin in predicting preeclampsia in a subject based on a comparison of a measurement of fibronectin in placenta-derived small extracellular vesicles of the subject's blood sample to at least one measurement of a control sample.

In some examples, an increase in the amount of fibronectin in the subject's blood sample relative to the control sample is predictive of preeclampsia.

Some examples further provide the use of JMJD6 in predicting preeclampsia based on a comparison of a measurement of JMJD6 in the placenta-derived small extracellular vesicles of the subject's blood sample to the at least one measurement of the control sample.

In further examples, a decrease in the amount of JMJD6 in the subject's blood sample relative to the control sample is predictive of preeclampsia.

In yet further examples, the preeclampsia is early-onset preeclampsia or late-onset preeclampsia.

In another aspect, the specification provides a method of predicting preeclampsia in a subject, the method including measuring an amount of fibronectin in placenta-derived small extracellular vesicles obtained from the subject, and comparing the amount of fibronectin to a control sample.

In some examples, the method includes isolating the placenta-derived small extracellular vesicles from a blood sample of the subject.

In further examples, the method includes measuring an amount of jumonji C domain containing protein 6 (JMJD6) and comparing the amount of JMJD6 to the control sample.

In some examples, if the measurements deviate statistically from the control sample, the subject is determined to be at risk of preeclampsia, and if the measurements do not deviate statistically from the control sample, the subject is not determined to be at risk of preeclampsia.

In further examples, a decrease in the amount of JMJD6 and an increase in the amount of fibronectin relative to the control sample is predictive of preeclampsia.

In further examples, isolating the placenta-derived small extracellular vesicles includes centrifuging the blood sample to obtain an isolate of small extracellular vesicles, and filtering the isolate to obtain the placenta-derived small extracellular vesicles.

In further examples, isolating the placenta-derived small extracellular vesicles includes immunoprecipitating the blood sample using a biotinylated anti-human placental alkaline phosphatase (PLAP) antibody.

In yet further examples, isolating the placenta-derived small extracellular vesicles includes sorting vesicles in the blood sample using a flow cytometer to obtain the placenta-derived small extracellular vesicles.

In other examples, measuring an amount of JMJD6 and measuring an amount of fibronectin includes conducting an enzyme-linked immunosorbent assay (ELISA) on the placenta-derived small extracellular vesicles.

In other examples, the blood sample is obtained from the subject between 10 and 34 weeks of gestation, and the method is for predicting early-onset preeclampsia.

In other examples, the blood sample is obtained from the subject after 34 weeks of gestation, and the method is for predicting late-onset preeclampsia.

In another aspect, the specification provides a kit for predicting preeclampsia. The kit includes a biotinylated anti-human placental alkaline phosphatase (PLAP) antibody for isolating placenta-derived small extracellular vesicles from a blood sample, and an anti-human fibronectin coating antibody for use in an enzyme-linked immunosorbent assay to measure an amount of fibronectin in the isolate of small extracellular vesicles.

In some examples, the kit includes an anti-human JMJD6 antibody for use in an enzyme-linked immunosorbent assay to measure an amount of JMJD6 in the isolate of small extracellular vesicles.

In some examples, the kit is used according to the above-described methods.

In another aspect, the specification provides a method of treating preeclampsia in a subject. The method includes isolating placenta-derived small extracellular vesicles from a blood sample obtained from the subject; measuring an amount of fibronectin in the placenta-derived small extracellular vesicles; comparing the amount of fibronectin to a control sample; and if the amount of fibronectin deviates statistically from the control sample, monitoring the subject for symptoms of preeclampsia.

In some examples, the method includes measuring an amount of jumonji C domain containing protein 6 (JMJD6) and comparing the amount of JMJD6 to the control sample.

In some examples, a decrease in the amount of JMJD6 and an increase in the amount of fibronectin relative to the control sample predicts preeclampsia.

In some examples, the method includes treating the subject with at least one of: hinokitiol, acriflavine, an antihypertensive drug, an anticonvulsant medication, a corticosteroid, an intravenous salt solution, a Caesarean section, induced delivery, and bed rest.

In some examples, isolating the placenta-derived small extracellular vesicles includes centrifuging the blood sample to obtain an isolate of small extracellular vesicles; and filtering the isolate to obtain the placenta-derived small extracellular vesicles.

In some examples, isolating the placenta-derived small extracellular vesicles further includes immunoprecipitating the blood sample using a biotinylated anti-human placental alkaline phosphatase (PLAP) antibody.

In some examples, isolating the placenta-derived small extracellular vesicles includes sorting vesicles in the blood sample using a flow cytometer to obtain the placenta-derived small extracellular vesicles.

In some examples, measuring the amount of JMJD6 and measuring the amount of fibronectin include conducting an enzyme-linked immunosorbent assay (ELISA) on the placenta-derived small extracellular vesicles.

In some examples, the blood sample is obtained from the subject between 10 and 34 weeks of gestation, and the method is for predicting early-onset preeclampsia.

In some examples, the blood sample is obtained from the subject after 34 weeks of gestation, and the method is for predicting late-onset preeclampsia.

In another aspect, the specification provides a kit for predicting preeclampsia. The kit includes a biotinylated anti-human placental alkaline phosphatase (PLAP) antibody for isolating placenta-derived small extracellular vesicles from a blood sample; and an anti-human fibronectin coating antibody for use in an enzyme-linked immunosorbent assay to measure an amount of fibronectin in the isolate of small extracellular vesicles.

In some examples, the kit includes an anti-human JMJD6 antibody for use in an enzyme-linked immunosorbent assay to measure an amount of JMJD6 in the isolate of small extracellular vesicles.

In some examples, the kit is used according to the above-described methods.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a flowchart showing a method of predicting preeclampsia.

FIG. 2 is a table showing the maternal and fetal clinical parameters of samples tested in Example 1.

FIG. 3A is a graph showing the representative NanoSight™ Nanoparticle Tracking Analysis of placental sEVs isolated at G1 (10-14 weeks) from maternal sera of normotensive control pregnancies.

FIG. 3B is a graph showing the representative NanoSight™ Nanoparticle Tracking Analysis of placental sEVs isolated at G1 (10-14 weeks) from maternal sera of late-onset preeclampsia pregnancies.

FIG. 4A is a Western blot for the placental sEV marker, PLAP, and the classical sEV markers, CD63 and TSG101, in sEVs isolated from maternal sera of control and pregnancies complicated with late-onset preeclampsia (L-PE).

FIG. 4B is a Western blot for fibronectin across 4 gestational windows (G1 to G4) in placental sEVs isolated from maternal sera in control pregnancies (n=14-19).

FIG. 5A is a graph showing the densitometry of FIG. 4B for fibronectin wherein fibronectin levels were normalized to total protein.

FIG. 5B is a graph showing the densitometry of FIG. 4B for fibronectin wherein fibronectin levels were normalized to sEV count.

FIG. 6 is a Western blot for fibronectin across G1 to G4 in placental sEVs in late-onset preeclampsia pregnancies (n=10-15).

FIG. 7A is a graph showing the densitometry of FIG. 6 for fibronectin across G1 to G4 in placental sEVs in late-onset pregnancies normalized to total sEV protein.

FIG. 7B is a graph showing the densitometry of FIG. 6 for fibronectin across G1 to G4 in placental sEVs in late-onset pregnancies normalized to sEV count.

FIG. 8 is a Western blot for fibronectin (FN) in circulating placental sEVs from control and late-onset preeclampsia (L-PE) pregnancies from G1 to G4 (n=15 controls and n=9 L-PE (late-onset preeclampsia) at G1, n=11-15 each for G2-G4).

FIG. 9A is a graph of the densitometry of FIG. 8 wherein fibronectin levels are normalized to total protein. 2-way ANOVA and Sidak posttest are shown.

FIG. 9B is a graph of the densitometry of FIG. 8 wherein fibronectin levels are normalized to sEV count. 2-way ANOVA and Sidak posttest are shown.

FIG. 10A is a Western blot for fibronectin and CD63 in total circulating sEVs from control and late-onset preeclampsia (L-PE) pregnancies from G1 to G4 (n=4 per group).

FIG. 10B is a Western blot for fibronectin and CD63 in nonplacental sEVs from control and late-onset preeclampsia (L-PE) pregnancies from G1 to G4 (n=4 per group).

FIG. 11A is a Western blot for fibronectin in circulating control and late-onset preeclampsia (L-PE) placental sEVs following treatment with trypsin. C1 and C2 are uncomplicated controls, (−) indicates untreated sEVs, and (+) indicates trypsin-treated sEVs.

FIG. 11B is a Western blot for FN following treatment of circulating placental sEVs with Peptide-N-Glycosidase F glycosidase enzyme.

FIG. 11C is a Western blot for concanavalin A (Con A) and fibronectin (FN) following immunoprecipitation of FN in circulating placental sEVs from control and L-PE pregnancies at G4 (n=4 per group). IgG=normal mouse negative control.

FIG. 12A is a Violin plot of sEV concentration in circulating placental sEVs from pregnancies complicated by E-PE (early-onset preeclampsia), L-PE (late-onset preeclampsia), or GH (gestational hypertension), compared with their respective age-matched term controls at G3 and G4. n=34 (controls at G3), n=17 (E-PE), n=33 (controls at G4), n=10 (L-PEat G4), n=13 (GH at G4), *P<0.05, **P<0.01, 1-way ANOVA and Tukey post-test.

FIG. 12B is a Violin plot of sEV size in circulating placental sEVs from pregnancies complicated by E-PE (early-onset preeclampsia), L-PE (late-onset preeclampsia), or GH (gestational hypertension), compared with their respective age-matched term controls at G3 and G4. n=34 (controls at G3), n=17 (E-PE), n=33 (controls at G4), n=10 (L-PE at G4), n=13 (GH at G4), *P<0.05, **P<0.01, 1-way ANOVA and Tukey post-test.

FIG. 13A is a Western blot for fibronectin normalized to PLAP protein in circulating placental sEVs from control and E-PE pregnancies at G1 (n=4 and 5, respectively).

FIG. 13B is a Western blot for fibronectin normalized to PLAP protein in circulating placental sEVs from control and E-PE pregnancies at delivery (G3, n=14 and 15, respectively).

FIG. 13C is a graph showing the densitometry of FIG. 13A. **P<0.01, unpaired Student t test.

FIG. 13D is a graph showing the densitometry of FIG. 13B. **P<0.01, unpaired Student t test.

FIG. 14A is a graph showing the densitometric analysis of fibronectin (FN) normalized to sEV count in circulating placental sEVs from control and E-PE pregnancies at G1. **P<0.01, ***P<0.001, Unpaired Student t test.

FIG. 14B is a graph showing the densitometric analysis of fibronectin (FN) normalized to sEV count in circulating placental sEVs from control and E-PE pregnancies at delivery (G3). **P<0.01, ***P<0.001, Unpaired Student t test.

FIG. 15A is a Western blot for FN in circulating placental sEVs from controls and pregnancies complicated by GH from G1 to G4 (n=7-10 per group),

FIG. 15B is a graph of the densitometry corresponding to FIG. 15A normalized to PLAP. Two-way ANOVA and Sidak post-test. Dashed lines on WBs indicate grouping of images from different parts of the same gel.

FIG. 15C is a graph of the densitometry corresponding to FIG. 15A normalized to sEV count. Two-way ANOVA and Sidak post-test. Dashed lines on WBs indicate grouping of images from different parts of the same gel.

FIGS. 16A to 16D are graphs showing the receiver operating curves (ROC) and corresponding area under the curve (AUC) values for fibronectin in circulating placental sEVs from G1 to G4 in L-PE pregnancies, normalized to sEV number, and plotted in terms of sensitivity and 1-specificity (both represented as a fraction of 1).

FIGS. 17A to 17D are graphs showing the receiver operating characteristic curves (ROCs) for fibronectin in circulating placental sEVs from G1 to G4 from pregnancies complicated by gestational hypertension, normalized to sEV number.

FIG. 18A is a Western blot for fibronectin in sEVs secreted by JEG3 cells maintained at either 3% O₂ or 21% O₂. Dashed lines indicate grouping of images from different parts of the same gel.

FIG. 18B is a graph showing the densitometry of FIG. 18A (n=4 separate sEV isolations, normalized to CD63 protein. P<0.05, unpaired Student t test).

FIG. 19A is the immunofluorescence of fibronectin and CD63 in JEG3cells exposed to either 3% O₂ or 21% O₂.

FIG. 19B is a graph of the Pearson Correlation Coefficient colocalization analysis corresponding to FIG. 19A (n=3 separate experiments, minimum 3 images per experiment, * P<0.05, Unpaired Student t test).

FIG. 20A is a graph of the NanoSight™ analysis of sEVs isolated from JEG3 conditioned media following JMJD6 siRNA interference (siJMJD6).

FIG. 20B is another graph of the NanoSight™ analysis of sEVs isolated from JEG3 conditioned media following JMJD6 siRNA interference (siJMJD6).

FIG. 21A is a Western blot for the sEV markers CD63, ALIX, TSG101, and FLOT-2 in sEVs derived from JEG3 cells, scrambled.

FIG. 21B is a Western blot for FN in sEVs from JEG3 cells following siRNA knockdown of JMJD6.

FIG. 21C is a graph corresponding to FIG. 21B showing the fibronectin protein fold change normalized to CD63 (n=3 separate isolations, * P<0.05, unpaired Student t test, SS=scrambled).

FIG. 22A is a Western blot for JMJD6 in circulating placental sEVs from control and E-PE pregnancies at G1.

FIG. 22B is a graph showing the densitometry for FIG. 22A (n=5, normalized to PLAP protein).

FIG. 23A is a Western blot for JMJD6 in circulating placental sEVs from control and E-PE pregnancies at delivery (G3).

FIG. 23B is a graph showing the densitometry for FIG. 23A (G3; n=15 and 17, respectively, normalized to PLAP protein).

FIG. 24A is a graph showing the densitometry for FIG. 22A, normalized to sEV count.

FIG. 24B is a graph showing the densitometry for FIG. 23A, normalized to sEV count.

FIG. 25A is a graph showing hydroxylysine content in circulating placental sEVs from control and E-PE pregnancies at delivery (n=3 and 7, respectively), **P<0.01, unpaired Student t test.

FIG. 25B is a graph of iron content in circulating placental sEVs from control and E-PE pregnancies at delivery (n=10 and 8, respectively), ***P<0.01, unpaired Student t test.

FIG. 26A is a Western blot for fibronectin normalized to CD63 protein in sEVs derived from JEG3 cells exposed to hypoxia (3% O₂) in the presence and absence of 1 μM Hinokitiol.

FIG. 26B is a graph of the densitometry corresponding to FIG. 26A (n=3 separate experiments; * P<0.05, 1-way ANOVA and Tukey posttest)

FIG. 27A is a Western blot for fibronectin in sEVs from placental villous explants obtained from term control and E-PE pregnancies. Dashed lines indicate grouping of images from different parts of the same gel.

FIG. 27B is a graph of the densitometry corresponding to FIG. 27A (n=3 placentae per group, each explant plated in triplicate; * P<0.05, 1-way ANOVA and Tukey posttest).

FIG. 28A is a representative immunofluorescence in JEG3 cells exposed to 21% O₂.

FIG. 28B is a representative immunofluorescence in JEG3 cells exposed to 3% O₂.

FIG. 28C is a representative immunofluorescence in JEG3 cells exposed to 3% O₂ and 1 μM Hinokitiol.

FIG. 28D is another representative immunofluorescence in JEG3 cells exposed to 21% O₂.

FIG. 28E is another representative immunofluorescence in JEG3 cells exposed to 3% O₂:

FIG. 28F is another representative immunofluorescence in JEG3 cells exposed to 3% O₂ and 1 μM Hinokitiol.

FIG. 29 is a graph showing Pearson's correlation coefficient colocalization analysis of fibronectin and CD63 in JEG3 cells exposed to 3% O₂ in the presence and absence of 1 μM Hinokitiol (n=3 separate experiments, minimum three images per experiment,* P<0.05, unpaired Student t test).

FIG. 30A is a single ROC curve for fibronectin (FN) in early-onset preeclampsia at G3, normalized to sEV count.

FIG. 30B is a multi-parameter ROC curve for fibronectin (FN)+JMJD6 in early-onset preeclampsia at G3, normalized to sEV count.

FIG. 30C is a single ROC curve for fibronectin (FN) in early-onset preeclampsia at G3, normalized to total control protein.

FIG. 30D is a multi-parameter ROC curve for fibronectin (FN)+JMJD6 in early-onset preeclampsia at G3, normalized to control protein.

FIG. 31 is a graph showing the fibronectin fold change in placental sEVs at G1, normalized for sEV number.

### DETAILED DESCRIPTION

### Table of Abbreviations

The following abbreviations are used herein:

| Abbreviation | Meaning |
|---|---|
| AUC | area under the curve |
| DAPI | 4′,6-diamidino-2-phenylindole |
| ED-B | extra domain B |
| ELISA | enzyme-linked immunosorbent assay |
| E-PE | early-onset preeclampsia |
| FN | fibronectin |
| G1 | 10-14 weeks of gestation |
| G2 | 6-22 weeks of gestation |
| G3 | 26-32 weeks of gestation |
| G4 | delivery |
| GH | gestational hypertension |
| JMJD6 | jumonji c domain containing protein 6 |
| L-PE | late-onset preeclampsia |
| NTA | nanoparticle tracking analysis |
| PE | preeclampsia |
| PLAP | placental alkaline phosphatase |
| pMSC | placental mesenchymal stromal cells |
| pMVECs | placental microvascular endothelial cell |
| PTC | pre-term control |
| ROC | receiver operating characteristic |
| sEV | small extracellular vesicle |
| SDS | sodium dodecyl sulfate |
| SDS-PAGE | sodium dodecyl sulfate-polyacrylamide gel electrophoresis |
| TC | term control |

### Definitions

The following definitions are used herein:

“Early-onset preeclampsia” herein refers to preeclampsia that develops before 34 weeks of gestation.

“Extracellular vesicle” herein refers to a particle previously known as an “exosome”, which is naturally released from the cell. Extracellular vesicles are delimited by a lipid bilayer and cannot replicate. Extracellular vesicles are defined by the Minimal Information for Studies of Extracellular Vesicles (“MISEV”) guidelines, proposed by the International Society for Extracellular Vesicles (ISEV), and published by the Journal of Extracellular Vesicles.

“Fibronectin” herein refers to a glycoprotein that comprises a major component of the extracellular matrix and contributes to cell adhesion, growth, migration and differentiation.

“Hinokitiol” herein refers to a monoterpenoid having the chemical structure

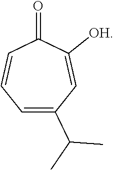

Hinokitiol is alternatively known as β-thujaplicin, beta-Thujaplicin, and 4-isopropyltropolone.

“JEG3” herein refers to a hypertriploid, clonally-derived, human cell line with epithelial morphology that was isolated from the Woods strain of the Erwin-Turner tumor.

“JMJD6” and “jumonji c domain containing protein 6” herein refer to a Fe(II)- and 2-oxoglutarate-dependent oxygenase that catalyses lysine hydroxylation and arginine demethylation of histone and non-histone peptides.

“Late-onset preeclampsia” herein refers to preeclampsia that develops after 34 weeks of gestation.

“Placental small extracellular vesicle” and “placenta-derived small extracellular vesicle” are used interchangeably herein to describe extracellular vesicles of placental origin.

“Small extracellular vesicle” herein refers to a subtype of extracellular vesicle, that measures between 50 nm and 200 nm in diameter.

### Methods of Predicting and Treating Preeclampsia

The methods will be described with respect to the figures herein.

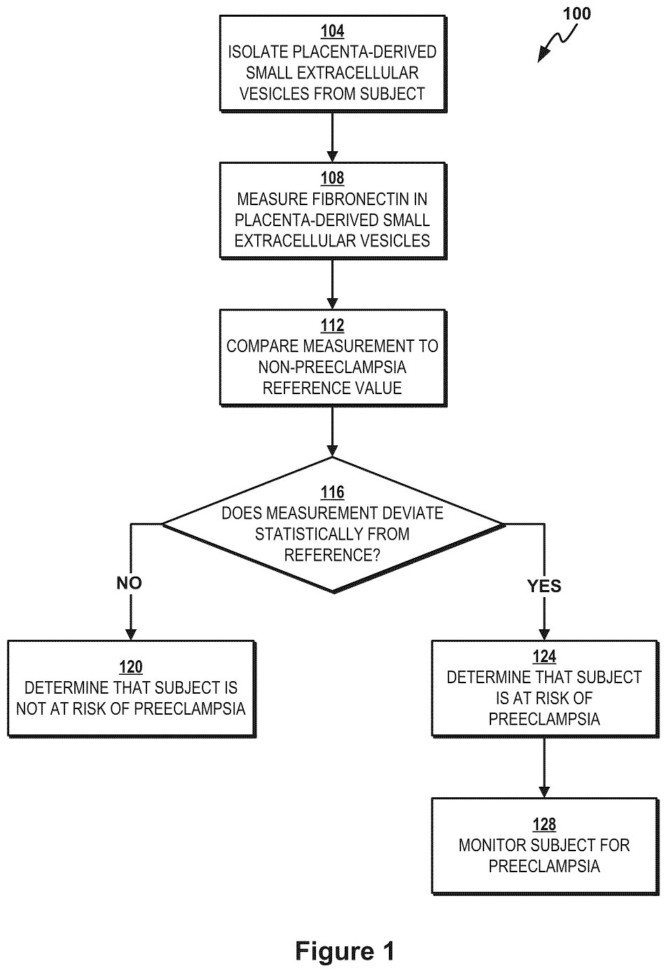

FIG. 1 is a flowchart showing a method 100 of predicting preeclampsia in a subject.

Block 104 comprises isolating placenta-derived small extracellular vesicles from a biological sample of the subject. The biological sample may comprise whole blood, plasma, serum, urine, saliva, cerebrospinal fluid, tissue biopsy, bone marrow, hair, nail clippings, stool, tears, sweat, synovial fluid, vaginal swab, placental tissue, amniotic fluid, fingernail or toenail clippings, skin biopsy, the like, or combinations thereof. In particular examples described herein, the biological sample comprises plasma. The placenta-derived small extracellular vesicles may be isolated from the biological sample using any suitable method or combination of methods including but not limited to, ultracentrifugation, differential centrifugation, filtration, gradient-based centrifugation, elution, microfluidics, magnetic separation technique, filtration, immunoprecipitation, Western blotting, fluorescence activated cell sorting (FACS), and flow cytometry.

In specific, non-limiting examples, the biological sample is differentially centrifuged and filtered to obtain a suspension of small extracellular vesicles, and immunoprecipitation is used to obtain the placental small extracellular vesicles from the suspension of small extracellular vesicles. The immunoprecipitation step may include incubating the suspension of small extracellular vesicles with an antibody specific to placental cells and immunoblotting the suspension to obtain an isolate of placental small extracellular vesicles. A specific non-limiting example of the antibody is a biotinylated anti-human placental alkaline phosphatase (PLAP) antibody. Another non-limiting example of the antibody is an anti-cytokeratin antibody.

In examples where the placental small extracellular vesicles are isolated from the biological sample with flow cytometry, a flow cytometer may be used to separate the placental small extracellular vesicles by size. The flow cytometer may be calibrated to identify and sort vesicles with a diameter corresponding to the diameter of a small extracellular vesicle. To isolate the placental small extracellular vesicles from the small extracellular vesicles, the flow cytometer may be configured to identify anti-PLAP antibodies. The anti-PLAP antibodies may be incubated with the suspension of small extracellular vesicles prior to sorting the vesicles. In other examples, the flow cytometer is used to isolate small extracellular vesicles by size, and immunoprecipitation is used to isolate the placental-derived extracellular vesicles.

As part of block 104, the small extracellular vesicles may be validated according to any suitable means known in the art. The sEVs may be validated by Western blotting, transmission electron microscopy (TEM), fluorescence-activity cell sorting (FACS), a particle analyzer, or a combination thereof. In examples where the small extracellular vesicles are validated with Western blotting, the Western blot may be used to identify a marker of small extracellular vesicles. In examples where the small extracellular vesicles are validated by a particle analyzer, the small extracellular vesicles may be validated with NanoSight™ analysis using a NanoSight™ NS 300 particle analyzer (Malvern Instruments Ltd, Malvern, UK) equipped with Nanoparticle Tracking Analysis (NTA) software.

Block 108 comprises measuring an amount of a biomarker in the placenta-derived small extracellular vesicles. The biomarker comprises fibronectin. The biomarker may further comprise jumonji C domain containing protein 6 (JMJD6). In particular, non-limiting examples, the biomarker is quantified with an enzyme-linked immunosorbent assay (ELISA), however block 108 is not particularly limited and the biomarker may be quantified with any suitable proteomics workflow known in the art. In other examples, the biomarker is quantified with Western blotting, mass spectrometry, Bradford assay, Lowry test, bicinchoninic acid assay, biuret method, ultra-violated visible (UV-vis) spectroscopy, or a combination thereof.

In examples where the biomarker is quantified with ELISA, an antibody specific to the biomarker may be used. In a specific, non-limiting embodiment, the biomarker may be quantified with an Invitrogen™ Fibronectin Human ELISA Kit (catalog #BMS2028, Thermo Fisher Scientific, Mississauga, Canada).

In some examples, the amount of the biomarker may be expressed relative to sEV count, the total amount of protein, or the amount of a control protein. In these examples, block 108 may further comprise measuring the sEV count, the total amount of protein, or the amount of the control protein. The control protein may include CD63, placental alkaline phosphatase (PLAP), or any other protein expressed in placenta-derived small extracellular vesicles.

Block 112 comprises comparing the measurement obtained at block 108 to measurements of a control sample representing a non-preeclamptic sample. The control sample may comprise a biological sample obtained from the subject at an earlier date, for example before pregnancy or at an earlier gestational stage. In some examples, the control sample comprises a biological sample obtained from a non-pregnant individual. In some examples, the control sample comprises a biological sample obtained from a non-preeclamptic individual. In further examples, the control sample comprises biological samples obtained from a plurality of non-preeclamptic individuals.

It should be generally understood that the biomarker is measured in the control sample using the same or similar methods as performed on the subject to obtain the measurement at block 108.

In examples where the control sample comprises a plurality of biological samples, the control measurement comprises a mean, average, standard deviation, minimum, or maximum measurement obtained for the plurality of biological samples.

Measurement of the control sample may be conducted prior to performance of method 100 to pre-determine a reference value. In these examples, block 112 comprises comparing the measurement obtained at block 108 to the reference value.

In specific non-limiting examples, the reference value comprises a JMJD6 fold change normalized to PLAP, and the JMJD6 fold change is between about 0.7 and about 1.5. In specific non-limiting examples, the reference value comprises a JMJD6 protein fold change normalized to the small extracellular vesicle count, and the JMJD6 fold change is between about 0.9 and about 1.2.

In specific non-limiting examples, the reference value comprises a fibronectin fold change normalized to the small extracellular vesicle count, and the fibronectin fold change is between about 0.9 and about 2.0. In specific non-limiting examples, the reference value comprises a fibronectin fold change normalized to the amount of PLAP, and the fibronectin fold change is between about 1.0 and about 2.0.

Block 116 comprises determining whether the measurement obtained at block 108 deviates statistically from the control sample for the biomarker.

The determination at block 116 includes determining whether the fibronectin content in the placental small extracellular vesicles of the subject exceeds the fibronectin content of the control sample. As will be described in greater detail in EXAMPLE 1, fibronectin content in placental small extracellular vesicles is elevated in preeclampsia.

In examples where the biomarker comprises JMJD6, the determination at block 116 includes determining whether the JMJD6 content in the placental small extracellular vesicles of the subject is lower than the JMJD6 content of the control sample. As will be described in greater detail in EXAMPLE 1, JMJD6 content in placental small extracellular vesicles is reduced in preeclampsia.

In order to determine whether the difference is statistically significant, any suitable statistical analysis may be applied including a two-factor ANOVA, Student t-test, Tukey post-test, post-test, and combinations thereof like.

If the determination at block 116 is “No”, the method may proceed to Block 120. Block 120 comprises determining that the subject is not at risk of preeclampsia or does not have preeclampsia.

If the determination at block 116 is “Yes”, the method may proceed to Block 124. Block 124 comprises determining that the subject is at risk of preeclampsia or has preeclampsia.

If the subject is determined to be at risk of preeclampsia, the method may proceed to block 128. Block 128 comprises monitoring the subject for preeclampsia. Monitoring the subject may comprise any suitable screening method known in the art, including but not limited to, blood pressure measurement, proteinuria, blood testing, fetal ultrasound, and symptom assessment.

Block 128 may further comprise treating the subject for preeclampsia. The treatment may comprise any suitable method known in the art including but not limited to, a therapeutically effective dose of hinokitiol, acriflavine, an antihypertensive drug, an anticonvulsant medication, a corticosteroid, or intravenous salt solution; Caesarean section or induced delivery; bed rest; or combinations thereof.

In examples where block 128 includes treating the subject with hinokitiol, the method further includes administering a therapeutically effective amount of hinokitiol to the subject to treat the preeclampsia. Hinokitiol prevents the hypoxic accumulation of fibronectin in small extracellular vesicles and restores fibronectin matrix deposition and turnover in preeclamptic placental mesenchymal stromal cells (pMSCs). Hinokitiol may be used prophylactically to treat the preeclampsia.

As part of block 128, the subject may be treated with a formulation comprising a therapeutically effective amount of hinokitiol. The formulation may comprise a synthetic hinokitiol or a natural extract of hinokitiol. In examples where the formulation comprises a natural extract, hinokitiol is extracted from Chamaecyparis sp., Thujopsis sp., or any other suitable organism. The formulation may further include one or more pharmaceutical excipients such as solvents, fillers, diluents, stabilizers, binders, suspension agents, viscosity agents, coatings, flavoring agents, disintegrants, colorants, lubricants, glidants, preservatives, sweeteners, propellants, anesthetic agents, the like, and combinations thereof. The formulation may comprise a cream, gel, lotion, ointment, shampoo, bodywash, soap, extract, tablet, capsule, liquid, or the like.

In view of the above, it will now be apparent that variant, combinations, and subsets of the foregoing embodiments are contemplated. For example, while method 100 was discussed above in relation to predicting preeclampsia, method 100 may be similarly used to diagnose preeclampsia or aid in the diagnosis of preeclampsia. Furthermore, method 100 may be used in combination with other screening parameters to assess a patient's risk of preeclampsia.

It will now be apparent to a person of skill in the art that the present specification affords certain advantages over the prior art. The use of placental small extracellular vesicles (sEVs) in the diagnosis of preeclampsia is advantageous because circulating placental sEVs are abundant during pregnancy and can be isolated inexpensively using basic proteomic methods. Furthermore, fibronectin and JMJD6 levels in placental sEVs can be used to detect both late-onset and early-onset preeclampsia, allowing for the development and use of prophylactic therapies.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

The methods will now be explained by way of example.

### Example 1

### 1. Materials and Methods

### Patient Population and Study Approval

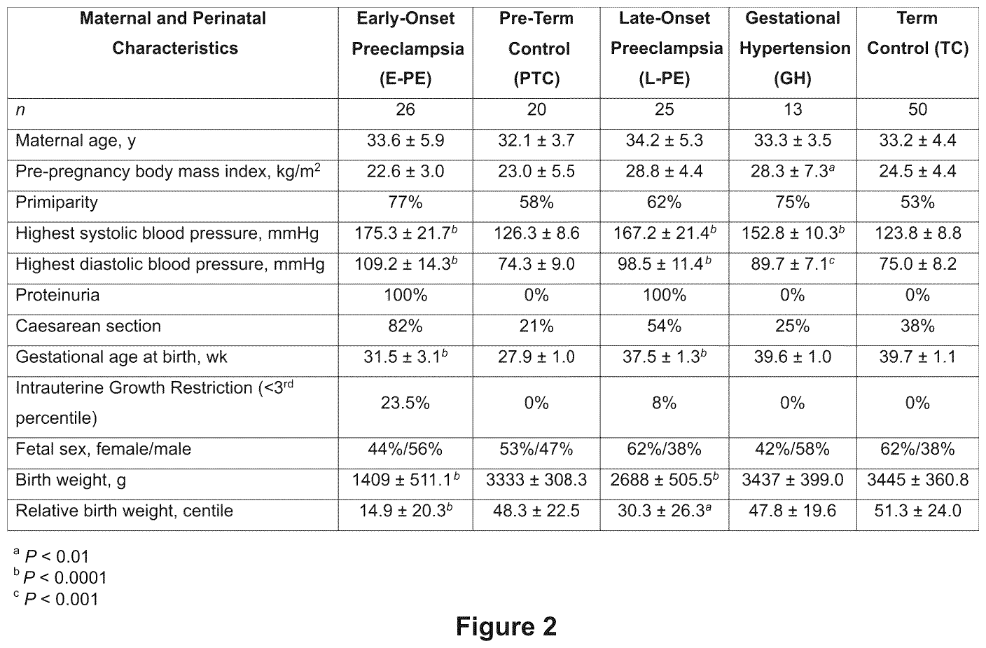

Procedures were conducted according to regulations of The Code of Ethics of the World Medical Association (Declaration of Helsinki) and Ethics Guidelines outlined by the Mount Sinai Hospital Research Ethics Board (REB: 17-0040-E). Informed consent was obtained from each subject. Maternal sera were accessed from the Ontario Birth Study consisting of a large-scale longitudinal pregnancy cohort (http://www.ontariobirthstudy.ca/). Pregnant women were recruited at their Nuchal Translucency Ultrasound or first prenatal visit. Maternal blood was collected at three visits during pregnancy (G1 herein refers to samples collected at 10-14 weeks of gestation, G2 herein refers to samples collected at 16-22 weeks of gestation, and G3 herein refers to samples collected at 26-32 weeks of gestation) and at delivery (G4 herein refers to samples collected at delivery). Hypertensive disorders of pregnancy were defined according to both the American College of Obstetricians and Gynecologists and the Society of Obstetricians and Gynecologists of Canada executive summary. Gestational hypertension (GH) refers to the isolated elevation of blood pressure after 20 weeks of gestation, whereas preeclampsia (PE) is diagnosed following hypertension and proteinuria, and in the absence of proteinuria, one or more adverse conditions or severe complications of pregnancy. Maternal and fetal clinical parameters are detailed in the table shown in FIG. 2 (PTC=preterm control; TC=term control). In FIG. 2, values are expressed as mean+standard deviation for continuous variables. Categorical variables expressed as proportion of subjects, as a percentage. Analysis was completed by Student's t test: E-PE vs PTC, L-PE vs TC, and GH vs TC.

SEV Isolation from Maternal Plasma and JEG3 Cell Conditioned Media

Total sEVs were isolated from maternal plasma (only frozen once at collection) by differential centrifugation and filtration, as described in Ermini, L., Ausman, J., Melland-Smith, M. et al. A Single Sphingomyelin Species Promotes Exosomal Release of Endoglin into the Maternal Circulation in Preeclampsia. Sci Rep 7, 12172 (2017). https://doi.org/10.1038/s41598-017-12491-4, the contents of which are incorporated herein by reference. For isolation of placental sEVs, 250 μL of total sEV suspension was incubated overnight at 4° C. with 3 μg of a biotinylated anti-human PLAP antibody (RRID:AB_10984896, SciCrunch, Research Resource Resolver).

JEG3 cells (RRID:CVCL_0363, SciCrunch, Research Resource Resolver) were grown to 60-70% confluency in 6-well plates at 37° C. in ambient air. For treatments, cells were exposed for 24 hours to 3% O₂ in the presence and absence of 1 μM Hinokitiol (diluted in DMSO; D8418, Millipore Sigma, Burlington, MA, USA) in EMEM containing 10% sEV-depleted fetal bovine serum (FBS; A2720803, Gibco, Thermo Fisher Scientific, Waltham, MA, USA) and 1% penicillin/streptomycin (Pen Strep; LS15140122, Gibco). sEVs were isolated from conditioned media via an ultrafiltration method using Amicon® Ultra-15 Centrifugal Filter Units (UFC901024, Millipore Sigma, Burlington, MA, USA) according to manufacturer's instructions. sEVs were validated by Western blotting for sEV markers and NanoSight™ analysis using a NanoSight™ NS 300 particle analyzer (Malvern Instruments Ltd, Malvern, UK) equipped with Nanoparticle Tracking Analysis (NTA) software.

### Placental Villous Explant Culture

Villous explants were isolated and cultured in 12-well plates with mesh inserts, from term control (n=3) and E-PE placentae (n=3). Term explants were maintained at normoxia (8% O₂) or hypoxia (3% O₂), while E-PE explants were maintained at 3% O₂ (constituting their normoxia) and treated with 2 M Hinokitiol. Following incubation with their respective treatments, explant tissue was snap-frozen in liquid nitrogen for protein extraction and downstream analyses, while small extracellular vesicles (sEVs) were isolated from conditioned media by ultrafiltration using Amicon® Ultra-4 Centrifugal Filter Units (UFC8100, Millipore Sigma).

### Antibodies

Primary antibodies employed in immunofluorescence and Western blotting include rabbit polyclonal anti-ALIX (RRID:AB_2637865, SciCrunch, Research Resource Resolver), mouse monoclonal anti-beta Actin (ACTB) (RRID:AB_11149557, SciCrunch, Research Resource Resolver), rabbit polyclonal anti-Calnexin (RRID:AB_10002123, SciCrunch, Research Resource Resolver), mouse monoclonal anti-CD63 (RRID:AB_10847220, hyperlinked to SciCrunch, Research Resource Resolver), mouse monoclonal anti-FN (RRID:AB_627598, SciCrunch, Research Resource Resolver), rabbit polyclonal anti-ED-B FN (RRID:AB_2921213, SciCrunch|Research Resource Resolver), rabbit polyclonal anti-FN (RRID:AB_2262874, SciCrunch, Research Resource Resolver), mouse monoclonal anti-FLOT2 (RRID:AB_627615, SciCrunch, Research Resource Resolver), rabbit polyclonal anti-JMJD6 (RRID:AB_1280968, SciCrunch, Research Resource Resolver), rabbit polyclonal anti-PLAP (RRID:AB_10900125, SciCrunch, Research Resource Resolver), mouse monoclonal anti-RAB7 (RRID:AB_10987863, SciCrunch, Research Resource Resolver) and recombinant rabbit monoclonal anti-TSG101 (RRID:AB_2809740, SciCrunch, Research Resource Resolver) antibodies. Secondary antibodies for Western blotting include goat anti-rabbit IgG-HRP (RRID:AB_631748, SciCrunch, Research Resource Resolver) and goat anti-mouse IgG-HRP (RRID:AB_631736, SciCrunch, Research Resource Resolver). For immunofluorescence experiments, secondary antibodies include Alexa Fluor® 488 donkey anti-rabbit IgG (RRID:AB_2535792, SciCrunch, Research Resource Resolver) and Alexa Fluor® 594 donkey anti-mouse IgG (RRID:AB_141633, SciCrunch, Research Resource Resolver), both obtained from Thermo Fisher Scientific™. Western Blots were scanned using a CanoScan™ LiDE20 image scanner (Canon Canada Inc. Mississauga, ON).

### Western Blotting and Immunofluorescence

For Western blotting, protein concentrations were quantified by a Bradford assay, and sEV samples containing 10-20 μg of protein in radioimmunoprecipitation assay (RIPA) buffer and 4×SDS sample buffer (250 mM Tris-HCl (pH 6.8), 8% (w/v) SDS, 40% (v/v) glycerol, 0.2% (w/v) bromophenol blue and 20% (v/v) β-mercaptoethanol) were subjected to 10% SDS-PAGE. Subsequent Western blotting was performed. For immunofluorescence analyses, JEG3 cells were grown to a confluency of ˜60% in 6-well plates on coverslips, following the appropriate treatment. Cells were immediately fixed in 4% (v/v) formaldehyde and immunofluorescence was performed.

### JMJD6 RNAi Knockdown

For ribonucleic acid interference (RNAi) knockdown of JMJD6, JEG3 cells were cultured to 50-60% confluency in 100-mm petri dishes and transfected with 30 nM of JMJD6 Silencer® siRNA duplexes (ID:23290, 4392420, Thermo Fisher Scientific, Waltham, MA, USA) or alternatively, with Silencer® Negative Control siRNA (4390844, Thermo Fisher Scientific). Transfection was performed using jetPRIME® in vitro siRNA transfection reagent according to manufacturer's instructions (Polyplus-Transfection®, Illkirch-Graddenstaden, France).

### Placental sEV Treatments

For trypsinization experiments, small extracellular vesicle (sEV) samples containing 10-20 μg of protein were incubated with 0.25% (v/v) Trypsin (27250-018, GIBCO, Thermo Fisher Scientific) in a 1:2 ratio for 45 min at 37ºC, centrifuged at 100,000 g for 90 min at 4ºC, and the pellet was resuspended in RIPA buffer and 4×SDS sample buffer. Samples were resolved on a 10% SDS-PAGE gel and probed for fibronectin and CD63. PNGase treatment of 20 μg sEV protein was carried on according to manufacturer's instructions (P0704S, New England Biolabs, Ipswich, MA, USA).

### Quantification of sEV Iron Content

Intra-sEV iron content was measured using a colorimetric Iron Assay Kit (ab83366, Abcam Inc.). Briefly, 106 sEVs (concentration was determined using nanoparticle tracking analysis) were diluted in Iron Assay buffer at a 1:5 ratio and sonicated for five rounds of 15 s each. Fe²⁺ levels were detected by addition of an iron probe containing Ferene S, an iron chromogen, to the reaction. Absorbance was measured using a microplate reader at an optical density of 593 nm. A standard curve was generated and Fe²⁺ concentrations were plotted; iron content was normalized per 106 sEVs.

### Quantification of sEV Hydroxylysine Content

Hydroxylysine content in placental small extracellular vesicles (sEVs) obtained from maternal sera was analyzed using the Quickdetect™ Hydroxylysine enzyme-linked immunosorbent assay (ELISA) kit according to manufacturer's instructions (RRID: AB_2927707). Content was normalized to the number of sEVs.

### Densitometry and Statistics

Statistical analyses were performed using GraphPad™ Prism software (RRID:SCR_002798, version 9.0.0 for Windows, GraphPad™ Software, San Diego, CA, USA). Data are expressed as mean+standard error of the mean. When comparing two groups, an unpaired Student's t-test was used. When comparing three or more groups across a single variable, a one-way ANOVA (analysis of variance) followed by a Tukey post-hoc test was performed. While comparing two or more groups across two independent variables, a two-way ANOVA and Sidak multiple comparison post-hoc test was performed. Significance was denoted as *p<0.05, ** p<0.01, *** p<0.001, and **** p<0.0001. Densitometric analysis was conducted by normalizing target protein levels to either total protein by stain-free gels or small extracellular vesicle (sEV) count as determined by nanoparticle tracking analysis. Receiver operating characteristic (ROC) curves were constructed to assess fibronectin and JMJD6 performance in predicting preeclampsia, and area under the curve (AUC) measured diagnostic accuracy.

### 2. Results

Fibronectin Content in Placental sEVs is Markedly Elevated in Preeclampsia

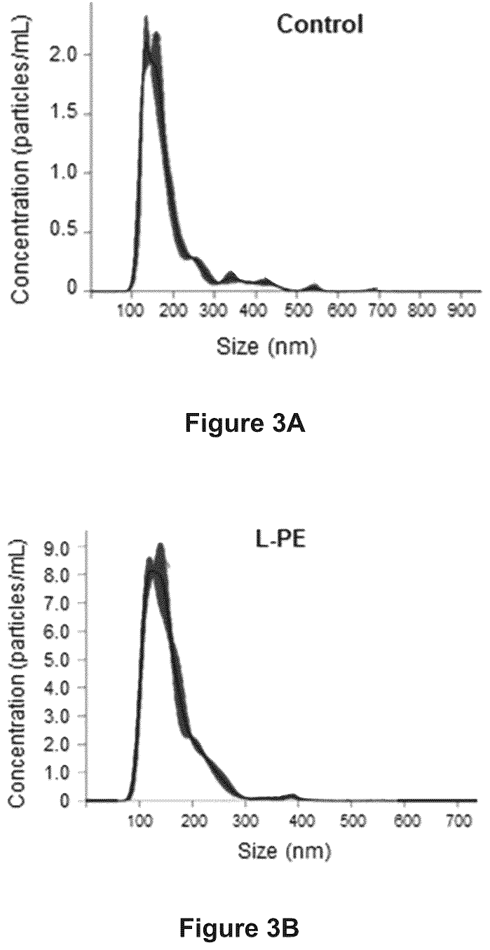

It was hypothesized that elevated placental and plasma fibronectin levels are associated with preeclampsia. It was examined whether placental small extracellular vesicles (sEVs) contribute to circulating fibronectin in the maternal bloodstream, and whether sEV fibronectin content is altered across gestation in various hypertensive disorders of pregnancy. Nanoparticle tracking analysis revealed a typical peak particle size at 100-150 nm, as shown in FIGS. 3A and 3B.

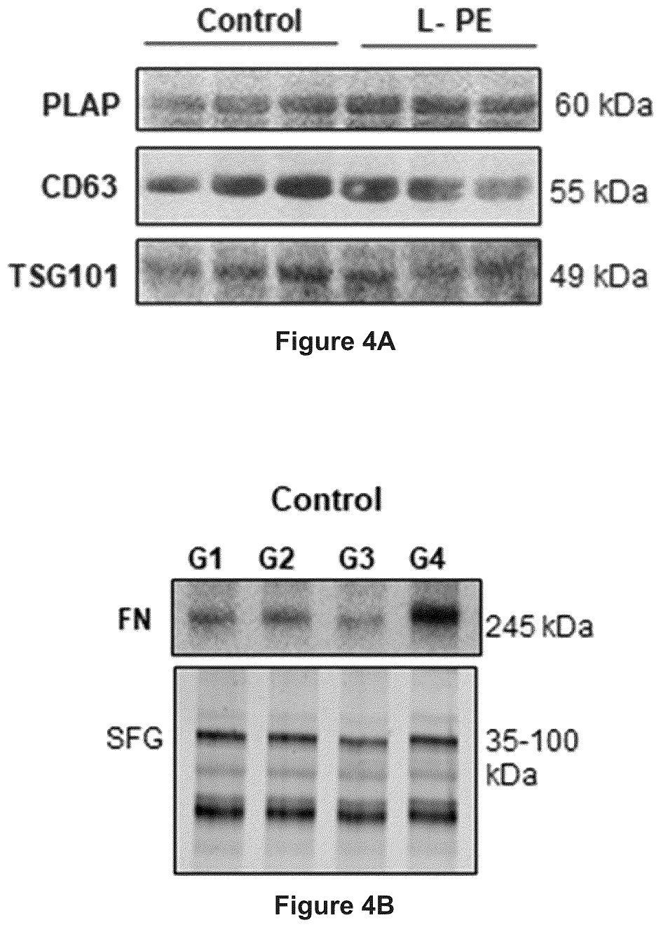

It is well-established that placental sEVs are uniquely marked by the presence of placental alkaline phosphatase (PLAP), an enzyme whose expression is largely restricted to the syncytium. A caveat is that low PLAP mRNA levels have been detected in other tissues. Nevertheless, highlighting their placental origin, PLAP⁺ sEVs are exclusively present in the peripheral circulation of pregnant individuals. Therefore, placental sEV quality was validated by positive Western blotting for PLAP, the transmembrane tetraspanin protein CD63, and tumour susceptibility gene 101 protein (TSG101) as well as negative Western blotting for ER marker calnexin (FIG. 4A).

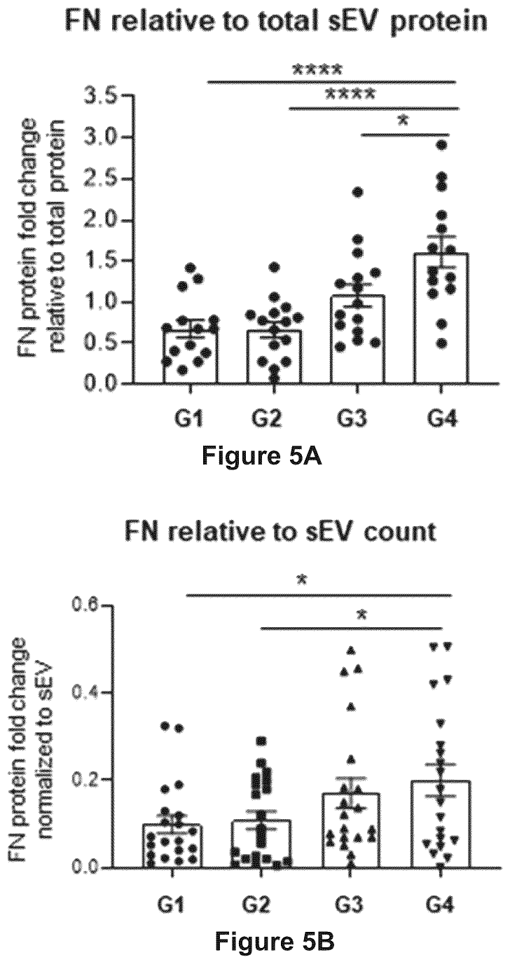

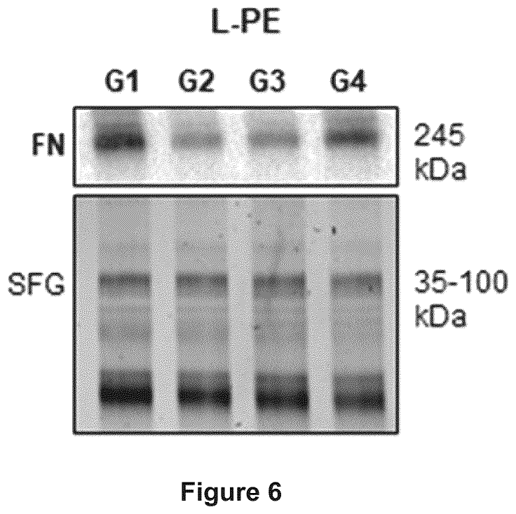

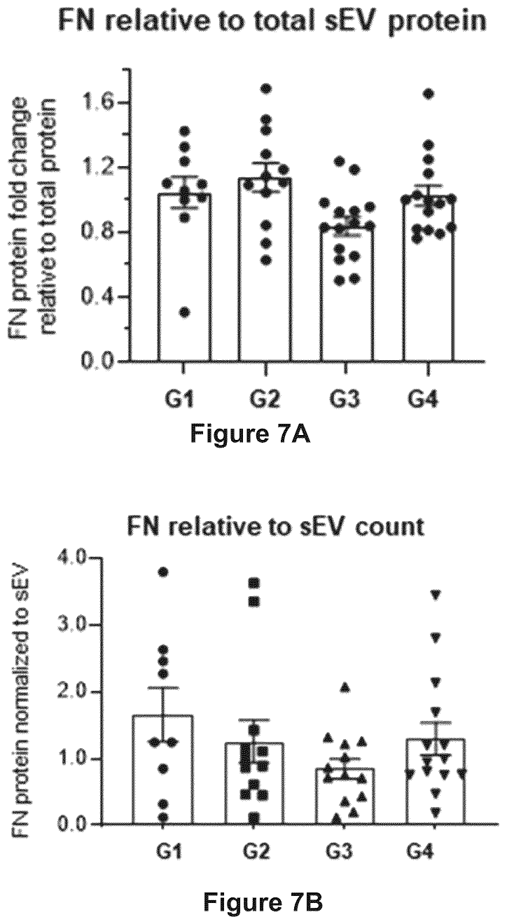

Next, fibronectin content was longitudinally examined in placental sEVs released in maternal sera of normotensive healthy control women and women with preeclampsia from G1 to G4. Western blotting for fibronectin in control placental sEVs revealed significantly elevated fibronectin levels at G4 relative to G1, G2 and G3 when fibronectin protein was normalized to total sEV protein levels (FIGS. 4B and 5A). Similarly, quantification of fibronectin protein relative to number of sEVs (i.e., particle count as determined by nanoparticle tracking analysis) revealed a significant increase at G1 and G2 vs. G4, as shown in FIG. 5B. In contrast, Western blotting for fibronectin in circulating non-placental sEVs (fraction of total sEVs in maternal plasma minus placenta-derived PLAP-positive sEVs) showed that sEV fibronectin is largely unchanged across G1 to G4. Interestingly, unlike in healthy control pregnancies, fibronectin levels were unchanged throughout gestation in circulating placental sEVs from L-PE pregnancies, whether fibronectin protein was normalized to total sEV protein or sEV number, as shown in FIGS. 6, 7A, and 7B.

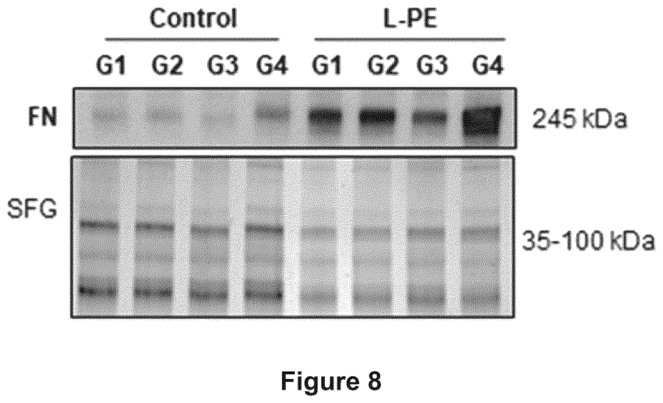

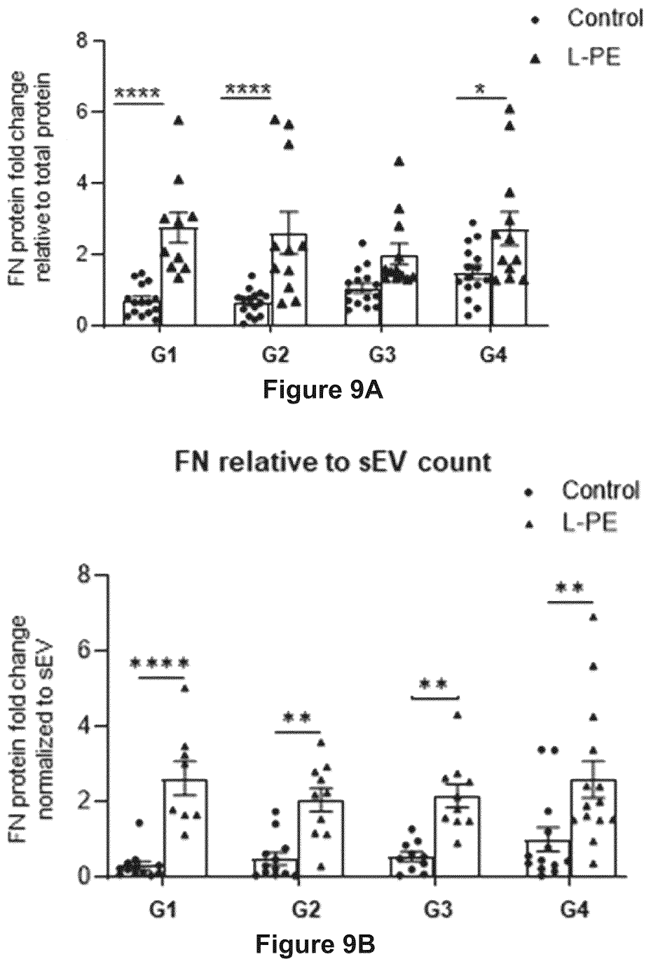

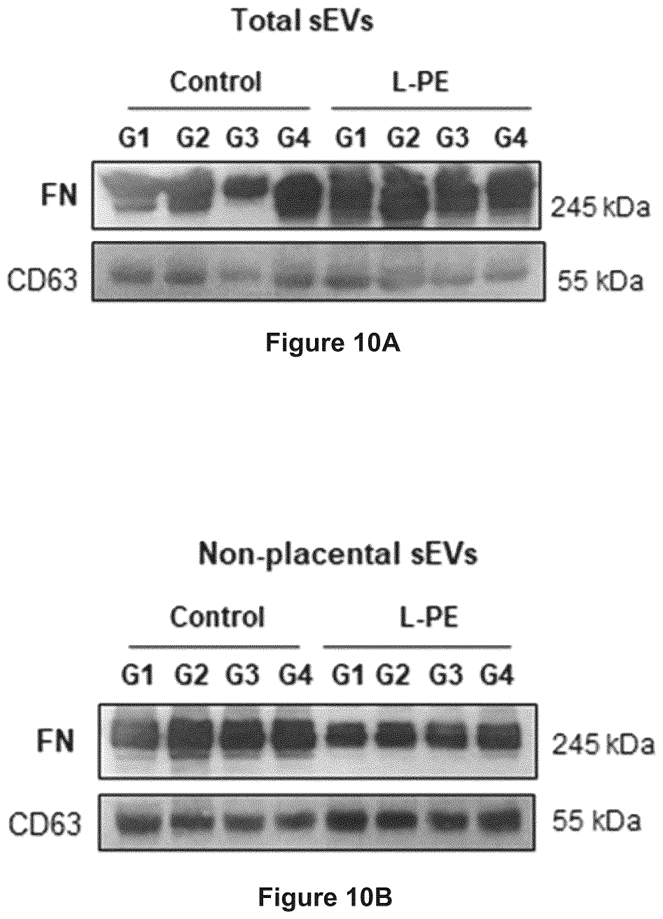

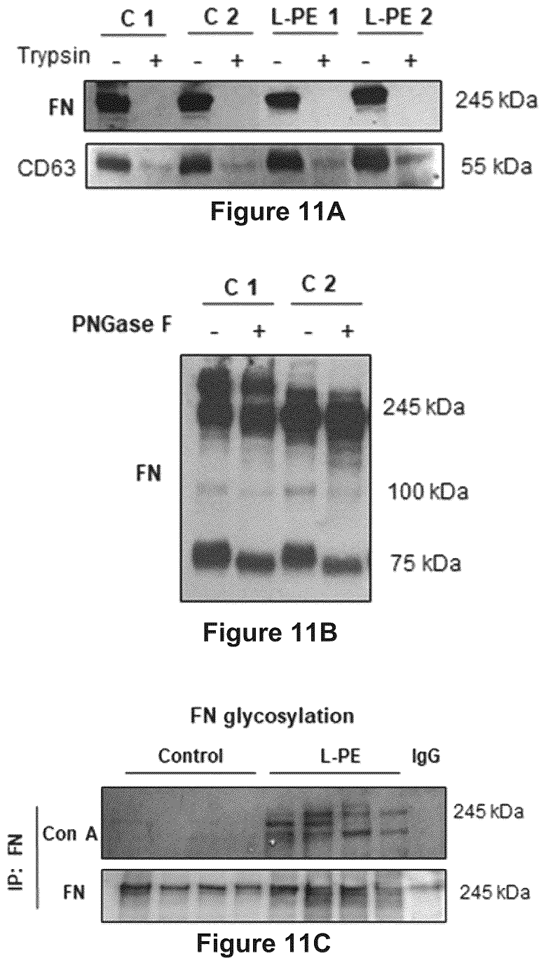

Direct comparison of sEV fibronectin in control and L-PE pregnancies revealed that fibronectin was significantly augmented in L-PE as early as G1 and continuing throughout gestation, whether it was quantified against total protein or sEV number (FIGS. 8, 9A, 9B). Notably, no significant differences were detected between control and L-PE pregnancies in total circulating sEVs (FIG. 10A), or in circulating non-placental sEVs (FIG. 10B). Exposure of placental sEVs to trypsin resulted in a loss of fibronectin (and CD63), indicating that sEV fibronectin predominantly resides on the surface of these EVs (FIG. 11A). Treatment of control and L-PE placental sEVs with Peptide-N-Glycosidase F (PNGase F), an enzyme that cleaves N-glycans from glycoproteins, and subsequent Western blotting for fibronectin identified three fragments of fibronectin (75, 100 and 245 kDa) that are glycosylated (FIG. 11B). Western blotting for the lectin concanavalin A (Con A) following immunoprecipitation of fibronectin revealed augmented signal in L-PE, indicative of enhanced glycosylation (FIG. 11C). Interestingly, no differences were observed in the expression of α5β1 integrin (the predominant fibronectin receptor) in placental sEVs in L-PE versus control sera, suggesting that the excess fibronectin in preeclampsia placental sEVs is bound to the membrane independent of its receptor. Pilot experiments incubating sEVs isolated from JEG3 cell-conditioned media with human plasma did not further increase the sEV fibronectin content.

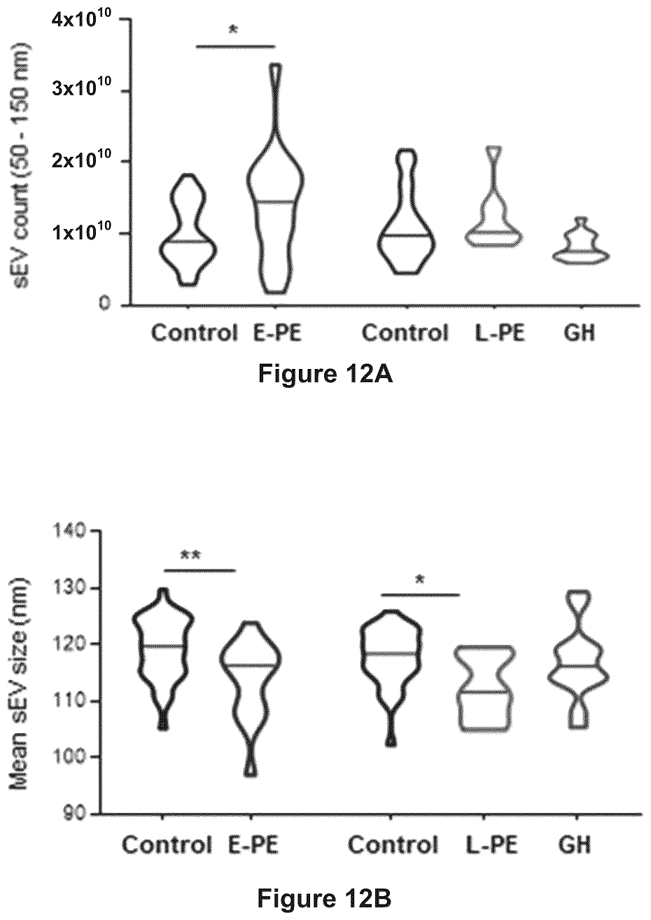

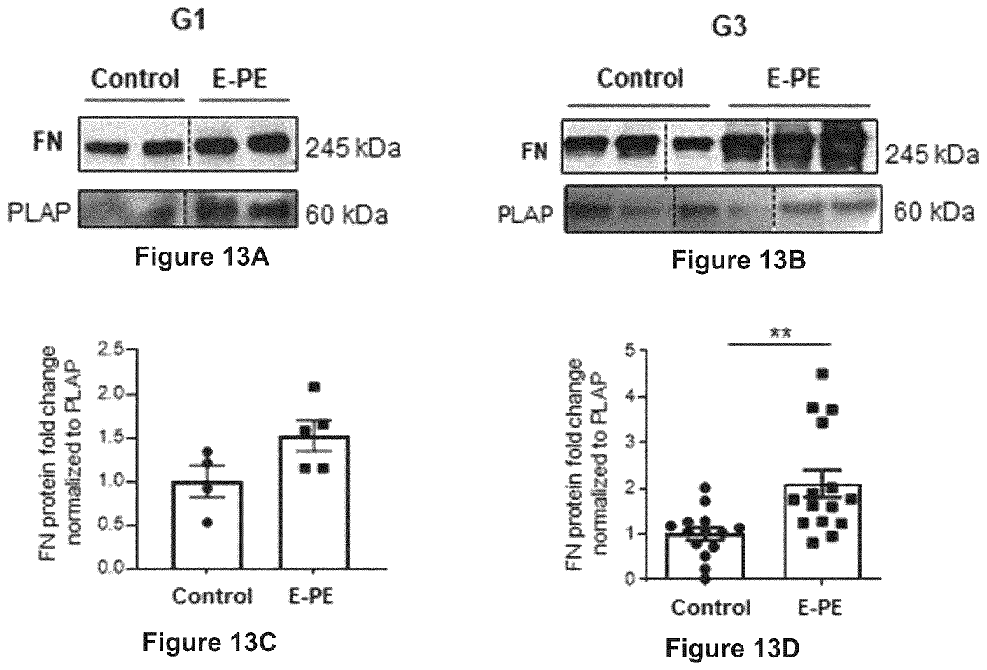

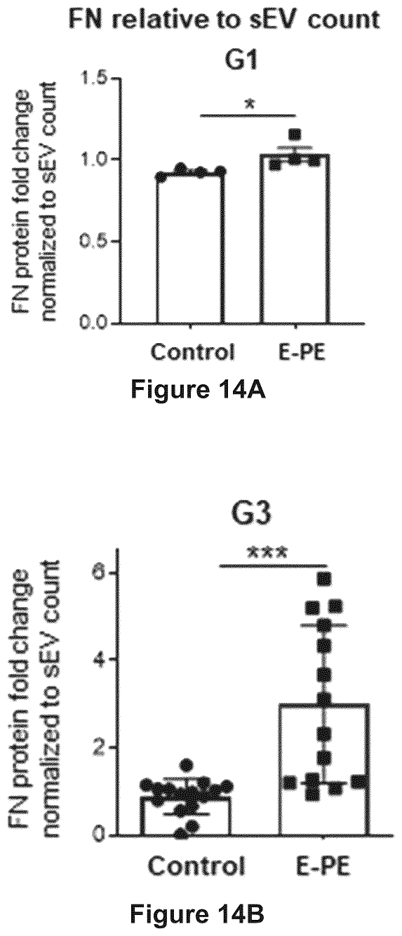

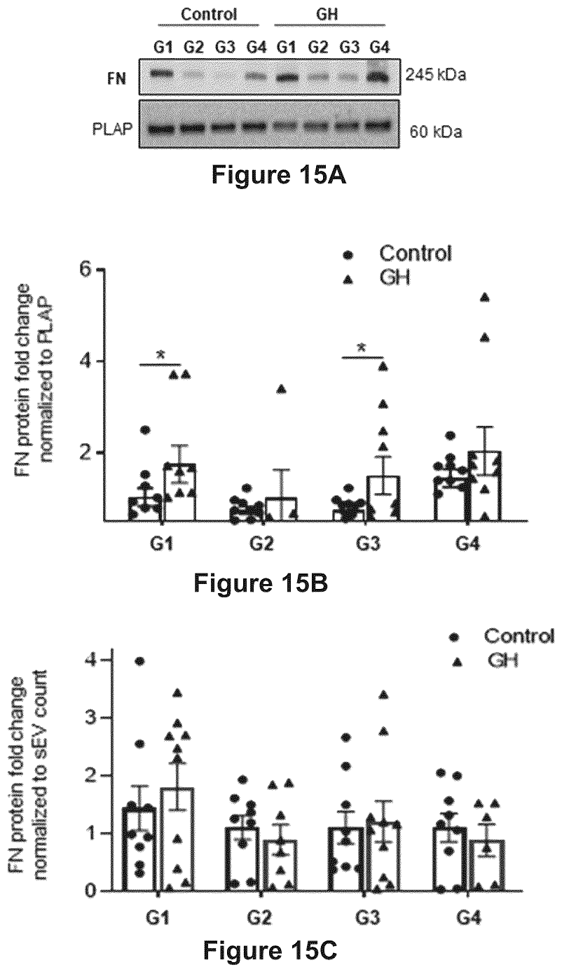

Given that preeclampsia is often distinctly defined as E-PE and L-PE based on age-of-onset and outcomes, fibronectin in placental sEVs in E-PE pregnancies at delivery (G3) was examined. Nanoparticle tracking analysis revealed a significant increase in placental sEV number in E-PE, while it was unchanged in L-PE and gestational hypertension (GH) in comparison to their respective controls (FIG. 12A). Mean sEV size was significantly smaller in E-PE and in L-PE relative to controls (at G3 and G4, respectively), while GH sEV size was unchanged (FIG. 12B). Elevated fibronectin content in E-PE placental sEVs was evident at both G1 (FIGS. 13A, 13C) and G3 (FIGS. 13C, 13D) independent of enhanced sEV number (FIG. 14A, 14B). This increase was unique to placental sEVs since a comparison of total circulating sEVs (including the placental fraction) at G3 revealed a significant decrease in fibronectin. In contrast, levels of onco-fetal fibronectin containing an extra domain B (ED-B FN) were unchanged in placental sEVs from control and L-PE pregnancies across gestation or from control and E-PE pregnancies at delivery respectively). Next, to determine whether the alterations in sEV fibronectin were exclusive to preeclampsia, fibronectin levels in placental sEVs from pregnancies complicated by gestational hypertension (GH) were compared to controls. Although fibronectin content was variable in GH sEVs, its levels relative to PLAP protein were significantly elevated in GH sEVs at G1 and G3, but not at G2 and G4 (FIGS. 15A, 15B). Following correction for sEV number, fibronectin did not significantly vary between control and GH pregnancies at any gestational age (FIG. 15C).

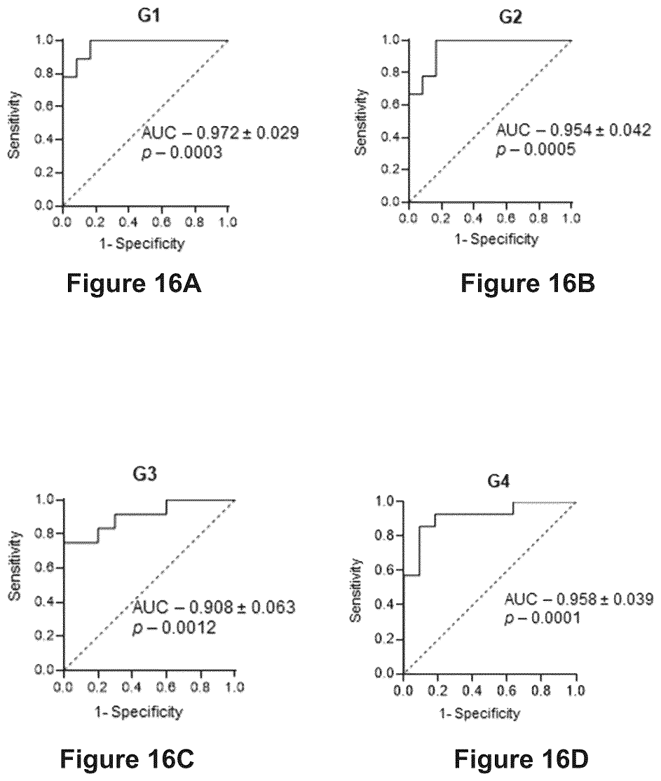

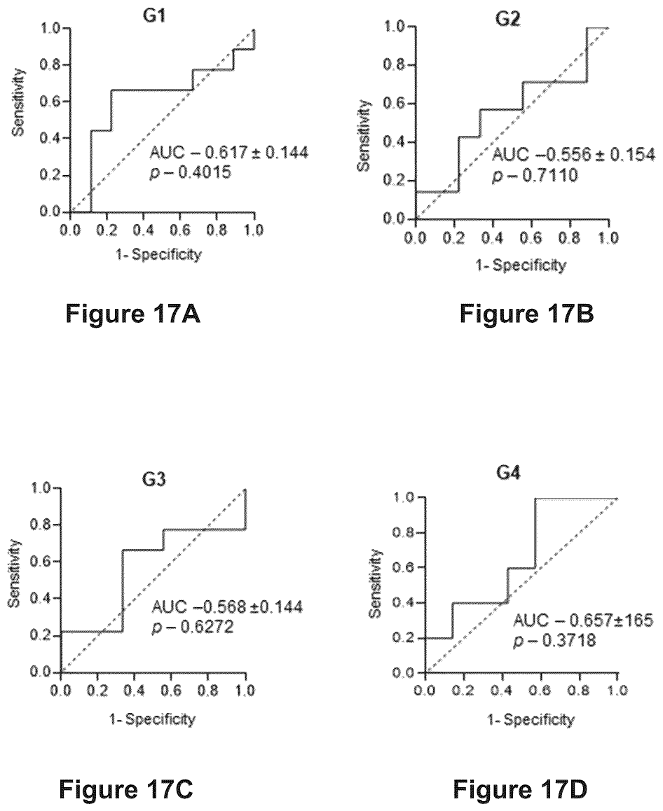

Lastly, to evaluate the diagnostic ability of placental sEV fibronectin to predict preeclampsia or gestational hypertension, Receiver operating characteristic (ROC) curves were constructed for fibronectin normalized to sEV number. The accuracy of fibronectin as a putative marker for late-onset preeclampsia was robust at all gestational time points, with highest AUC at G1 (0.972; FIGS. 16A to 16D). AUC remained low for GH throughout pregnancy (0.556 to 0.657; FIGS. 17A to 17D). ROC curves for fibronectin normalized to total protein in the two pathologies revealed a similar trend of high AUC for late-onset preeclampsia (0.797-0.987) and low values for gestational hypertension (0.575-0.780).

Hypoxia Stimulates Fibronectin in sEVs from Trophoblast Cells

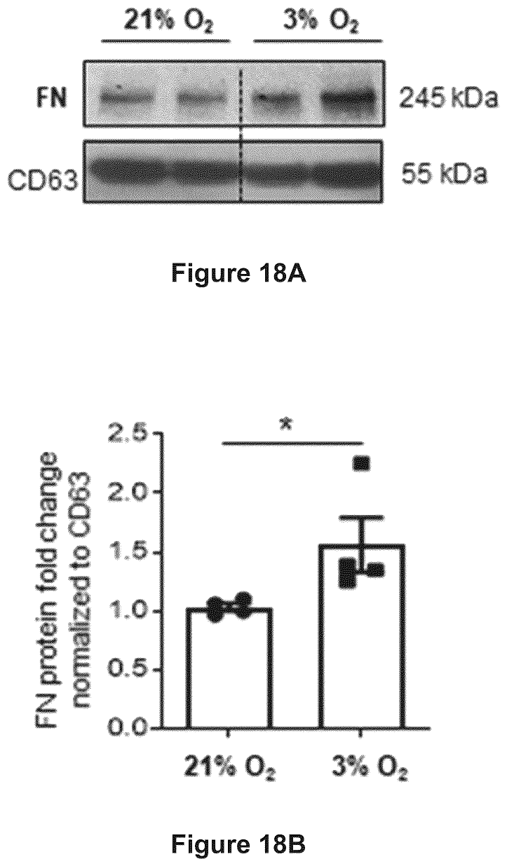

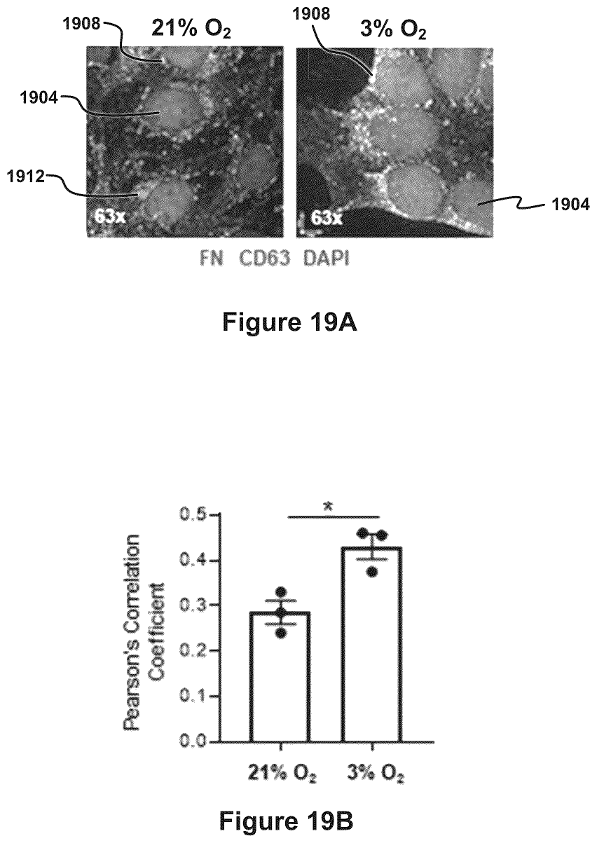

Hypoxia is a potent regulator of extracellular matrix-associated proteins, including fibronectin. It was investigated whether hypoxia, a characteristic of preeclampsia placentae, contributed to the striking changes in sEV fibronectin in preeclampsia. Western blotting for fibronectin in small extracellular vesicles (sEVs) isolated from conditioned media from JEG3 cells maintained in hypoxia (3% O₂) revealed that low oxygen exposure significantly stimulated fibronectin release via sEVs (FIG. 18A, 18B). Next, the sEVs were assessed to determine whether low oxygen facilitated the movement of fibronectin into sEVs prior to exiting the cell. Immunofluorescence and corresponding Pearson's correlation coefficient analysis showed that fibronectin increasingly co-localized with CD63 in cells maintained at 3% O₂ (FIGS. 19A and 19B). In FIG. 19A, 1904 indicates DAPI (4′,6-diamidino-2 phenylindole), 1908 indicates fibronectin, and 1912 indicates CD64. Similarly, hypoxic exposure promoted fibronectin co-localization with flotillin-2 (FLOT2), a plasma membrane protein that is enriched in sEVs.

The Oxygen Sensor, JMJD6, is Depleted in Placental sEVs from Preeclampsia Pregnancies

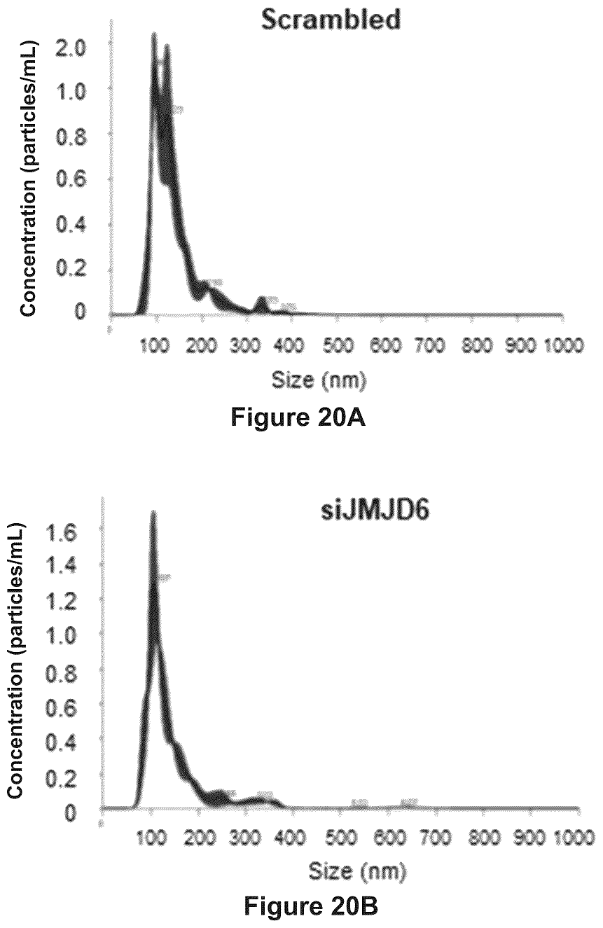

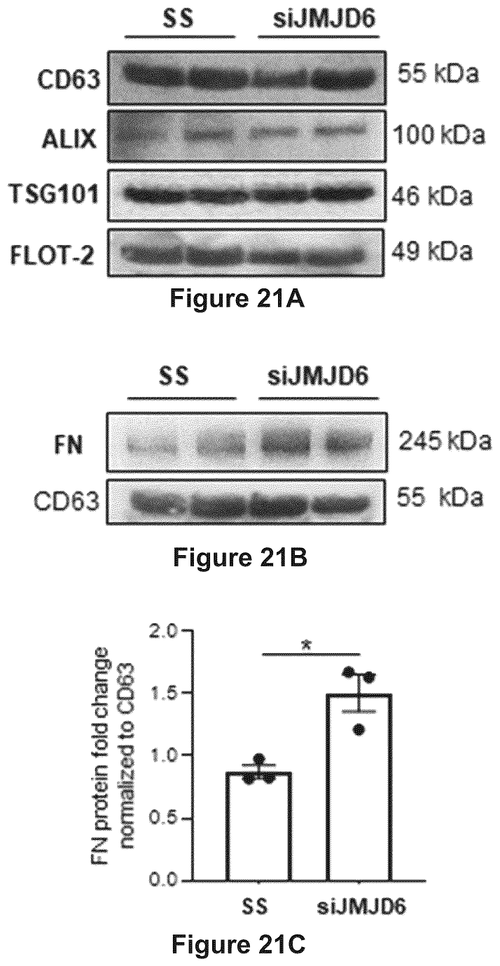

Considering that the oxygen- and iron-dependent lysyl hydroxylase JMJD6 is a negative regulator of fibronectin, it was examined whether altered JMJD6 expression and function may also impinge on sEV fibronectin in preeclampsia. Following siRNA knockdown of JMJD6 in JEG3 cells, attenuation of JMJD6 levels was corroborated by Western blotting for JMJD6, while nanoparticle tracking analysis (FIGS. 20A, 20B) and Western blotting for sEV markers CD63, ALIX, TSG101 and FLOT-2 (FIG. 21A) validated the sEVs released. Importantly, JMJD6 loss resulted in significantly elevated fibronectin in JEG3-derived sEVs (FIGS. 21B, 21C), suggesting that JMJD6 is required for maintenance of steady state levels of fibronectin in trophoblast cells and in sEVs.

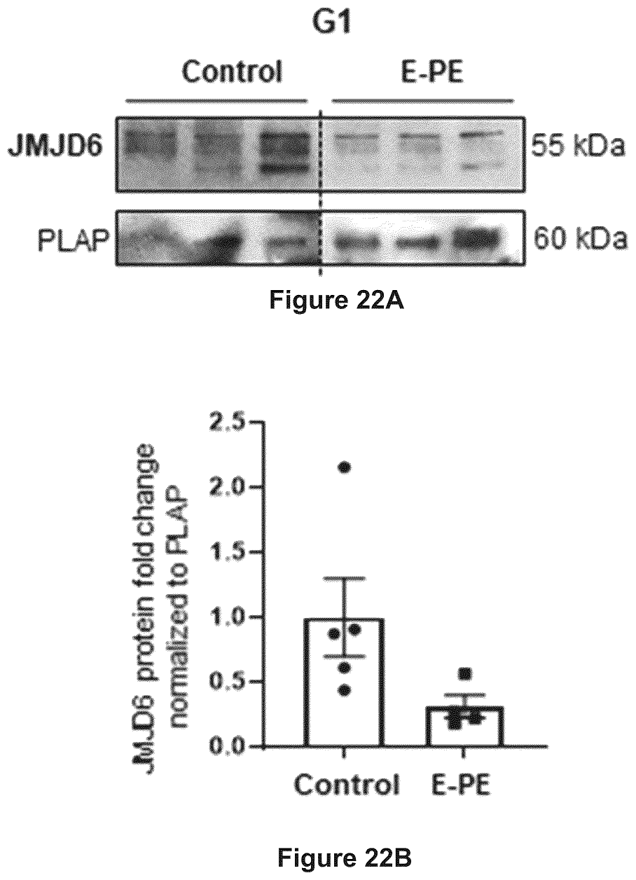

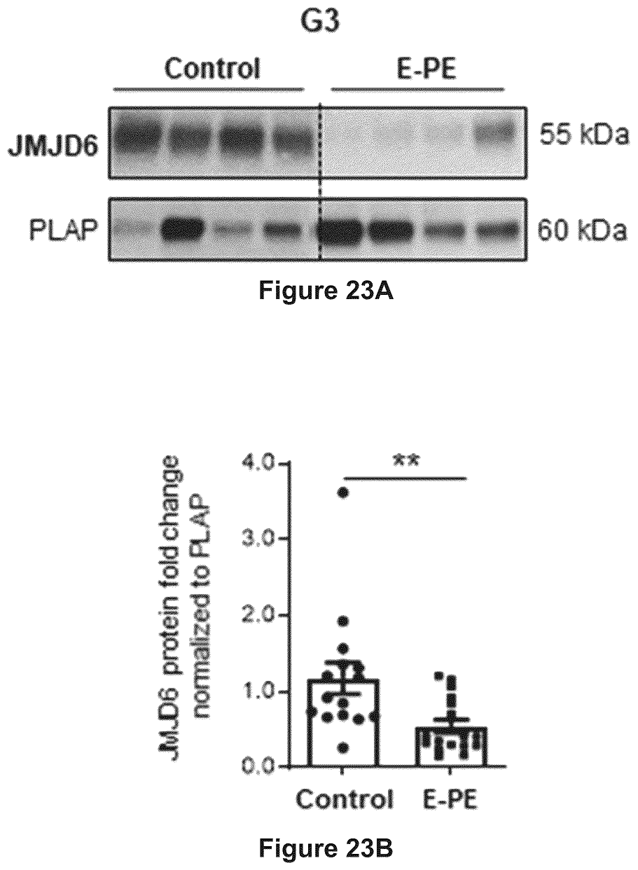

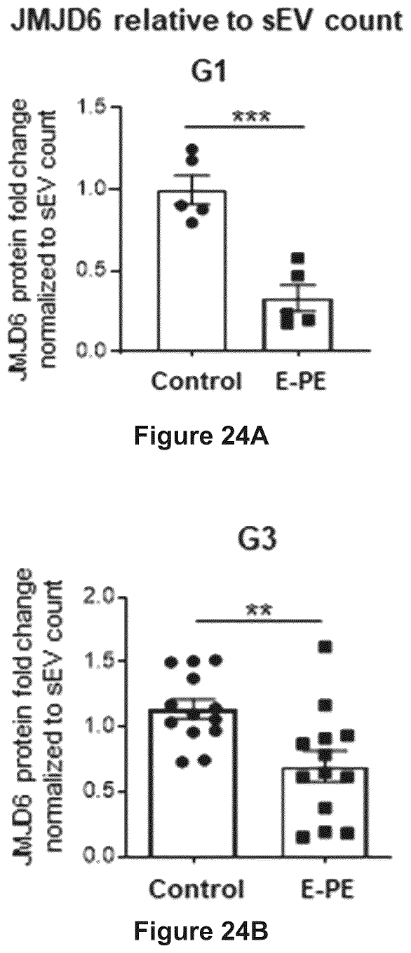

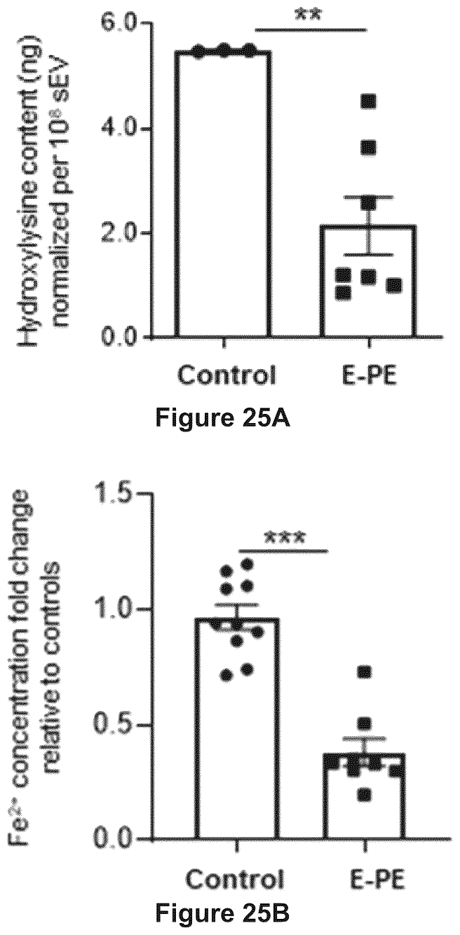

Intriguingly, JMJD6 was present in placental sEVs; however, its content significantly diminished in sEVs from E-PE at both G1 and G3, whether it was normalized relative to PLAP levels (FIGS. 22A, 22B, 23A, 23B) or sEV number (FIGS. 24A, 24B). This was accompanied by a marked reduction in sEV hydroxylysine content (a readout for JMJD6 lysyl hydroxylase activity) in E-PE at G3, implying reduced JMJD6 function (FIG. 25A). Ferrous iron (Fe²⁺) is a critical co-factor for JMJD6 enzyme function, and preeclampsia placenta display reduced Fe²⁺, partly contributing to impaired JMJD6 activity. Fe²⁺ content was markedly decreased in E-PE placental sEVs at G3 compared to their respective age-matched controls (FIG. 25B). JMJD6 protein levels were significantly decreased in L-PE placental sEVs when normalized to PLAP levels but remained unchanged relative to sEV count.

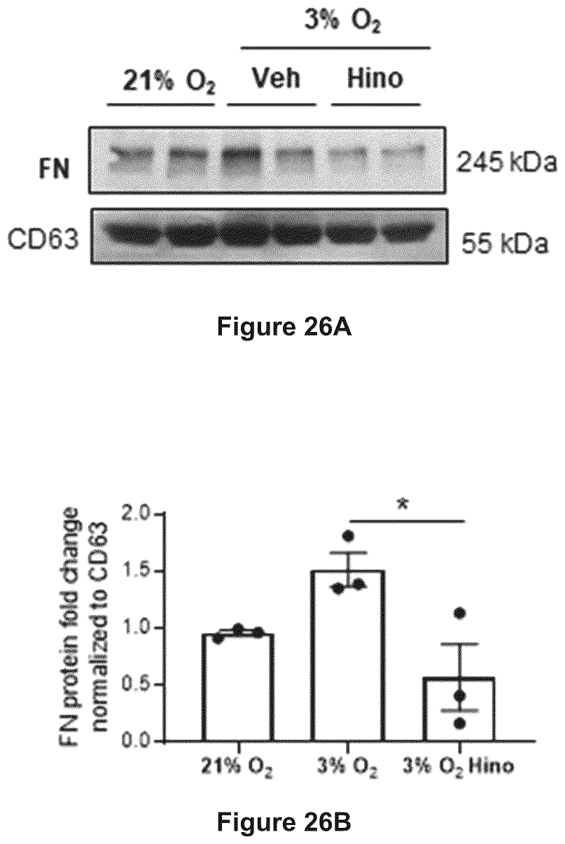

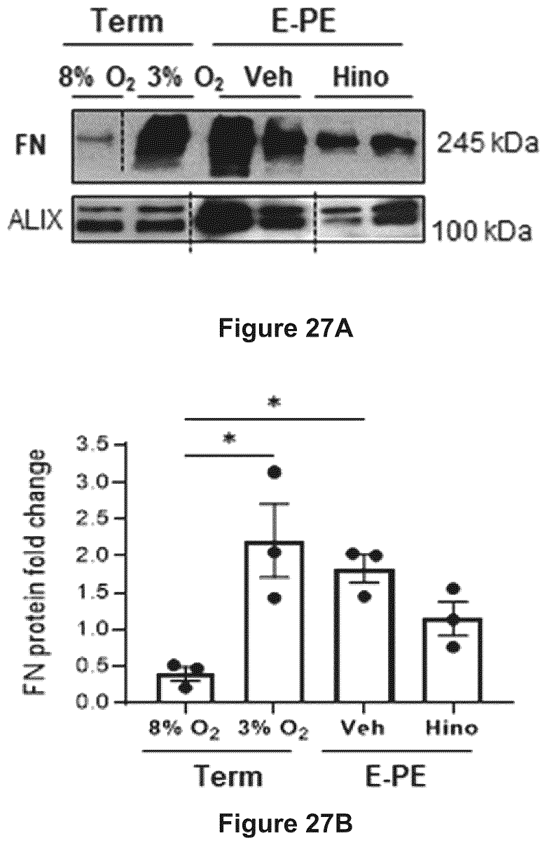

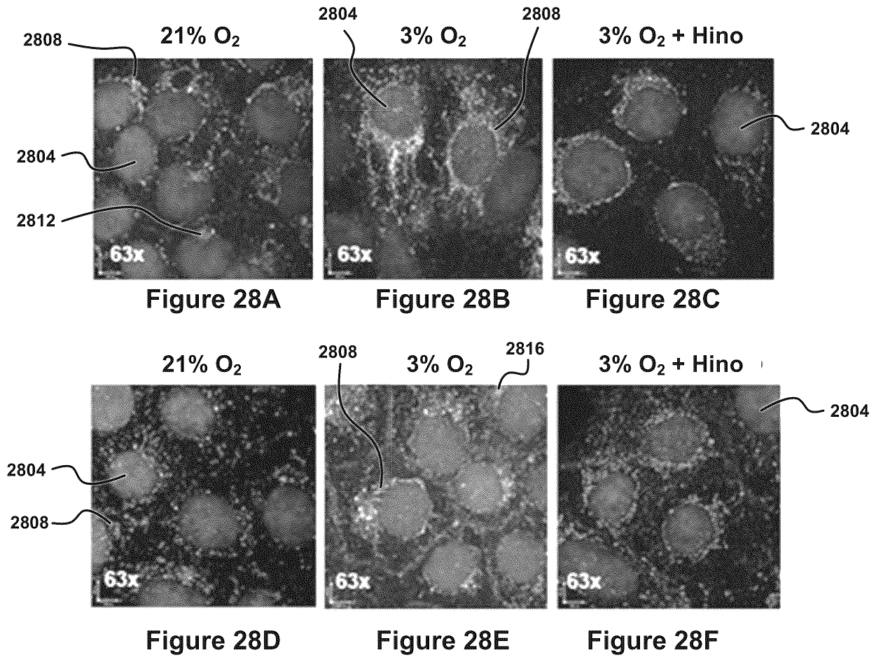

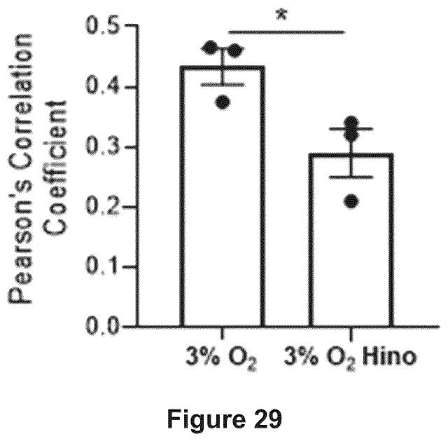

Exposure of preeclampsia pMSCs to the natural compound, Hinokitiol restored the labile (useable) intracellular iron pool and improved fibronectin fibrillar deposition. Treatment of JEG3 cells maintained in 3% O₂ with 1 μM Hinokitiol significantly reduced sEV fibronectin content to control levels (FIGS. 26A, 26B). In contrast, Hinokitiol treatment in 21% 02 had no discernible effect on sEV fibronectin levels. In line with the JEG3 data, exposure of villous explants from term placentae to low oxygen (3% O₂) enhanced fibronectin in explant-derived sEVs compared to control explants maintained at physiological (8%) O₂ (FIGS. 27A, 27B). This fibronectin increase was recapitulated in sEVs from E-PE villous explants maintained at 3% O₂, which was attenuated by addition of Hinokitiol (FIGS. 28A to 28F). In FIGS. 28A to 28F, 2804 indicates DAPI, 2808 indicates fibronectin, 2812 indicates RAB7, and 2816 indicates CD63. Immunofluorescence analysis revealed that Hinokitiol addition attenuated fibronectin co-localization with the late endosomal marker RAB7 and the sEV marker CD63, relative to JEG3 cells in hypoxia alone (Pearson's Correlation Coefficient between fibronectin and CD63, of 0.29 for 21% O₂, 0.40 for 3% O₂ and 0.31 for 3% O₂+Hinokitiol; FIG. 29).

Elevated Fibronectin and Diminished JMJD6 in Circulating Placental sEVs are a Signature for Early-Onset Preeclampsia

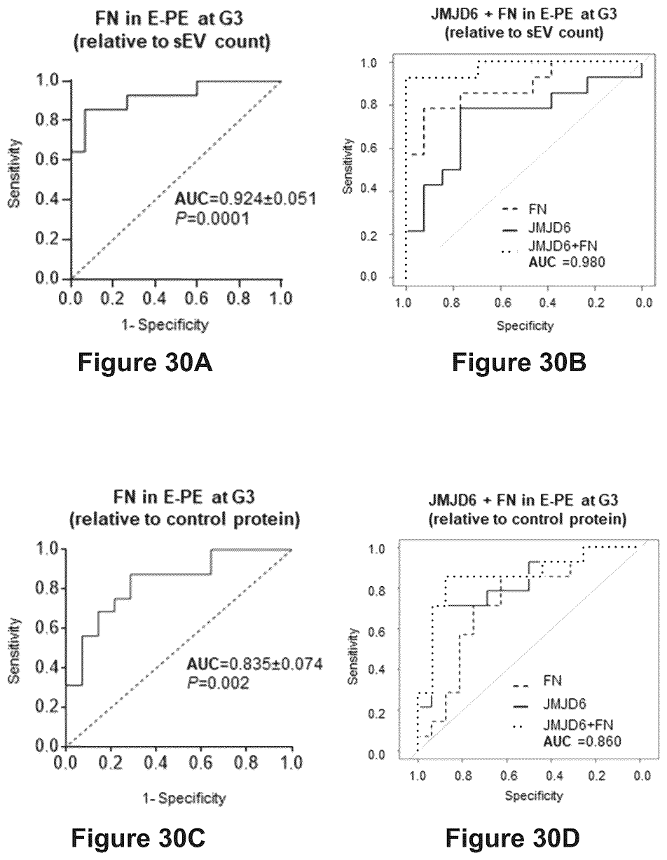

The contrasting changes in fibronectin in small extracellular vesicles (sEVs) and JMJD6 protein in E-PE led us to explore their potential predictive value. While fibronectin exhibited a high AUC value (0.924) individually when normalized to sEV count, multivariate ROC curve analysis for fibronectin in combination with JMJD6 revealed a further enhanced AUC of 0.980 for E-PE at G3 (FIGS. 30A, 30B). A similar trend was observed when individual fibronectin and multivariate fibronectin+JMJD6 ROC curves were constructed upon normalization to total sEV protein (i.e., AUC=0.835 for fibronectin and AUC=0.860 for fibronectin+JMJD6; FIGS. 30C, 30D).

### 3. Discussion

PE is a devastating syndrome that is notoriously challenging to predict. The present study identifies sEV fibronectin as a promising putative marker for the early detection of preeclampsia. Placental sEVs derived from both early- and late-onset preeclampsia pregnancies display strikingly elevated fibronectin content as early as the first trimester of gestation, much prior to the onset of maternal clinical symptoms. This is accompanied by a pronounced reduction in sEV content and function of JMJD6, an iron-dependent oxygen sensor and lysyl hydroxylase, in E-PE pregnancies. Its cellular impairment due to hypoxia and Fe²⁺ deficiency may underlie sEV fibronectin excess, particularly in E-PE. Supporting this concept, hypoxic inhibition of JMJD6 function induced fibronectin secretion via sEVs in trophoblast cells while exposure of trophoblast cells to Hinokitiol, a natural compound that restores intracellular Fe²⁺, attenuated hypoxia-induced sEV fibronectin release by restoring cellular JMJD6 function.

Elevated Fibronectin Content in Maternal Circulating sEVs Predicts Preeclampsia Onset

Given the ubiquitous nature of fibronectin, it is not surprising that aberrant fibronectin levels are implicated in a host of pathologies including vasculopathies, heart and liver disease, renal fibrosis, and cancer. Of note, studies have determined that circulating plasma fibronectin may be an important biomarker in pregnancy-related pathologies such as gestational diabetes mellitus (GDM) and intrauterine growth restriction. In addition, the premature presence of onco-fetal fibronectin in cervico-vaginal secretions of pregnant women between 21 and 36 weeks has historically been used as a predictive marker for spontaneous pre-term birth. While other groups have detected increased fibronectin in maternal plasma in pregnancies destined to develop preeclampsia, much of the research on the utility of fibronectin as a biomarker has focussed on detecting the glycosylated plasma form (secreted by hepatocytes) and the cellular onco-fetal form, neither of which are exclusively a reflection of placental function. As such, large-scale systematic reviews have concluded that plasma fibronectin concentrations may not be clinically useful in predicting the risk of developing preeclampsia. Example 1 shows for the first time that fibronectin is robustly detected in circulating placenta-derived sEVs throughout pregnancy, and that its content is markedly elevated in preeclampsia throughout gestation and beginning as early as 10-14 weeks. Interestingly, ED-B fibronectin, an alternatively spliced gene product, is present in placental sEVs, but is largely unchanged in early-onset preeclampsia (E-PE) or late-onset preeclampsia (L-PE), indicating that this isoform may not particularly reflect preeclampsia ‘stress’ during pregnancy. In contrast, the rise in circulating sEV fibronectin in preeclamptic pregnancies is primarily due to placenta-derived sEVs carrying total full-length cellular fibronectin. In parallel, an overall increase in placental sEV number in the maternal circulation of E-PE pregnancies at delivery was observed. Despite this, fibronectin protein levels in E-PE were strikingly elevated, suggesting that altered sEV production does not directly account for changes in circulating sEV fibronectin in preeclampsia.

A current tool for aiding the diagnosis of preeclampsia involves examining the ratio of serum levels of soluble fms-like tyrosine kinase 1 (sFlt-1) to placental growth factor (PIGF) which are anti- and pro-angiogenic factors respectively. Despite their reliability in diagnosing preeclampsia, their ability to predict the disease early on (i.e., first trimester) is debatable. Given the fundamental role of the placenta in perpetuating preeclampsia, particularly E-PE, examining placenta-derived sEVs paints a more comprehensive picture of disease progression. Moreover, the use of trophoblast-derived fibronectin in placental sEVs provides an advantage since circulating placental sEVs during pregnancy are abundant and can be isolated using straightforward methods. The first trimester of pregnancy is a critical gestational window coinciding with establishment of a mature placenta and feto-maternal blood flow that is postulated to go awry in women that go on to develop preeclampsia. Hence, targeting this important period for assessing biomarkers, in combination with other early screening parameters, will permit the development of prophylactic therapy for this complex disorder. Interestingly, fibronectin content in placental sEVs from gestational hypertension pregnancies had low predictive value. Gestational hypertension is distinct from preeclampsia, and hence, the putative diagnostic ability of fibronectin in preeclampsia with much higher accuracy than gestational hypertension suggests that elevated fibronectin in placental sEVs is predominantly a placental affair.

New evidence highlights sEVs as carriers of specific ECM components, including fibronectin, leading to fibril deposition and the potential to remodel matrix architecture and guide directional cell movement. sEVs secreted by preeclampsia pMSCs have enriched fibronectin cargo, directly impacting HTR-8/SVneo EVT migratory capacity. Example 1 shows that fibronectin is predominantly captured onto the surface of placental sEVs, like tetraspanins that have been shown to associate with integrin receptors. In support of this, fibronectin was found to reside exofacially in sEVs derived from lung fibroblasts as well as trophoblasts. Lung fibroblast-derived sEV fibronectin in turn mediated cell invasion, while that from human trophoblasts stimulated the production of the pro-inflammatory cytokine, interleukin-1B (IL-1B) by macrophages. Furthermore, sEV fibronectin from myeloma cells activated downstream mitogen-activated protein kinases (MAPK) signaling pathways in recipient cells upon anchoring to heparan sulfate. Hence, it is plausible that depending on cellular origin (even within the placenta), placental sEV fibronectin can mediate distinct cellular events in the maternal circulation upon reaching target organs. In particular, the endothelial dysfunction that is typical of preeclampsia may in part stem from the direct interaction of excessive hyper-glycosylated fibronectin with maternal vasculature, further contributing to maternal clinical symptoms.

### Loss of JMJD6 in E-PE May Contribute to Excessive sEV Fibronectin Release

An emerging body of evidence emphasizes the role of sEVs in the transport and transfer of microRNAs (miRNAs) to target cells, given their ability to regulate gene expression. The involvement of other genetic and epigenetic modifiers, particularly of histone modifying enzymes, has largely been overlooked. JMJD6, a multi-faceted histone demethylase and lysyl hydroxylase, is abundantly present in sEVs secreted by JEG3 trophoblastic cells and in circulating placental sEVs. The data further suggest that the presence of ferrous iron in sEVs permits JMJD6 to autonomously function, as it does in cells. Given the diversity of JMJD6 targets, its presence in sEVs has significant implications not only for the extracellular control of its protein targets (such as fibronectin), but also for the potential to modify recipient cells' genome. Under pathological conditions (as in early-onset preeclampsia), significantly reduced placental sEV iron content can, therefore, directly inhibit JMJD6 activity within the sEVs, resulting in aberrant cargo transfer and signalling.

JMJD6 is critically involved in controlling fibronectin production and deposition in pMSCs of early-onset preeclampsia (E-PE) origin, which is disrupted due to hypoxia/iron imbalance. JMJD6 loss and concomitant increase of fibronectin in in the same subset of E-PE, but not L-PE placental sEVs not only underscores the regulatory relationship between the two factors, but also highlights another unique distinction between the two sub-pathologies. For instance, the finding that JMJD6 is significantly reduced in E-PE sEVs but not L-PE, is in line findings on compromised JMJD6 enzyme activity in E-PE placental tissue stemming from hypoxia-iron imbalance. Not surprisingly, the diagnostic accuracy of JMJD6 in E-PE sEVs at G3 is higher than that for L-PE (AUC 0.828 versus 0.704 respectively).

Prior to sEV secretion, fibronectin has been shown to co-localize with both early- and late-endosomal compartments that are the precursors to sEVs. The analysis of fibronectin trafficking in JEG3 cells revealed that hypoxia markedly promoted the association of fibronectin with CD63-positive multivesicular bodies (MVBs). This may in part be explained by an overall upregulation of cellular fibronectin production (likely due to hypoxic inhibition of JMJD6 activity) leading to augmented release as a way of disposal. On the other hand, it may be due to the hypoxia microenvironment stimulating sEV production, resulting in an enhanced release of fibronectin-containing sEVs.

Intriguingly, addition of the natural compound Hinokitiol prevented the hypoxic accumulation of fibronectin in sEVs from JEG3 cells and placental villous explants. Hinokitiol (alternatively known as β-thujaplicin) is a tropolone derivative that was initially recognized for its non-toxic, anti-inflammatory, and anti-tumour properties. It was recently implicated in modulating diverse cellular processes including restoring tissue iron balance across cellular gradients and anti-metastatic effects on cancer cells. No studies have reported that Hinokitiol has a direct impact on the amount of fibronectin released in sEVs. This complements the observation that Hinokitiol treatment of preeclampsia pMSCs restored fibronectin matrix deposition and turnover.

In conclusion, this study provides novel evidence on placental sEV-derived fibronectin and its regulator, JMJD6, as potential predictors for preeclampsia and its severity. A major strength of this study is the sizeable cohort of control and pathological cases across human pregnancy, spanning early first trimester to delivery. Furthermore, NanoSight™ nanoparticle tracking analysis was used to assess sEV number in every patient at different gestational time points, thereby permitting the normalization of fibronectin and JMJD6 according to appropriate sEV particle count. The findings described herein warrant larger-scale studies examining the utility of fibronectin and JMJD6 in preeclampsia at different gestational time points throughout pregnancy.

### Example 2

Measuring Fibronectin Content in Placental sEVs with ELISA

Placental sEVs were isolated from control subjects at G1 and late-onset preeclampsia (L-PE) subjects at G1, according to the methods described above in Example 1.

The number of small extracellular vesicles (sEV) in each sample was determined by nanoparticle tracking analysis.

Fibronectin levels were quantified in each sample using enzyme-linked immunosorbent assays (ELISA). An anti-human fibronectin coating antibody was used to immobilize the fibronectin in a Corning™ Costar™ 96 Flat Bottom Transparent Polystyrene microplate (Thermo Fisher Scientific, Mississauga, Canada). The optical density of each sample was measured with a Tecan® Infinite 200 PRO (Tecan Life Sciences) and analyzed with Tecan i-Control™ software to determine the concentration of fibronectin.

### Results

Table 1 shows the results for control placental sEVs.

TABLE 1
Control placental sEVs (G1)

| | OD | OD | Avg | Concentration | Particle count | Particle count | FN normlzd | Fold |
|---|---|---|---|---|---|---|---|---|
| Sample | (1) | (2) | OD | (x = y − b/m); ng/mL | (per mL) | (per 1.5 uL) | per sEV # | change |
| 225C | 0.603 | 0.742 | 0.672 | 7.003 | 2.39E+08 | 358869.6 | 1.95E−05 | 2.649 |
| 869 | 0.927 | 0.737 | 0.832 | 9.091 | 6.56E+08 | 984560.3 | 9.23E−06 | 1.254 |
| 853 | 0.213 | 0.196 | 0.204 | 0.876 | 6.91E+08 | 1036581.3 | 8.46E−07 | 0.115 |
| 149C | 0.680 | 0.665 | 0.672 | 7.001 | 3.61E+08 | 541091.9 | 1.29E−05 | 1.757 |
| 845 | 0.522 | 0.478 | 0.500 | 4.745 | 4.87E+08 | 731171.5 | 6.49E−06 | 0.881 |
| 849 | 0.158 | 0.154 | 0.156 | 0.244 | 6.50E+08 | 974445.6 | 2.50E−07 | 0.034 |
| 857 | 0.256 | 0.234 | 0.245 | 1.412 | 6.06E+08 | 909223.9 | 1.55E−06 | 0.211 |
| 873 | 0.471 | 0.620 | 0.545 | 5.339 | 4.39E+08 | 659225.5 | 8.10E−06 | 1.100 |
| | | | | | | Cntrl Avg | 7.37E−06 | |

Table 2 shows the results for L-PE placental sEVs.

TABLE 2
L-PE placental sEVs (G1)

| | OD | OD | Avg | Concentration | Particle count | Particle count | FN normlzd | Fold |
|---|---|---|---|---|---|---|---|---|
| Sample | (1) | (2) | OD | (x = y − b/m); ng/mL | (per mL) | (per 1.5 uL) | per sEV # | change |
| 18 | 0.487 | 0.665 | 0.576 | 5.742 | 3.11E+08 | 465823.7 | 1.23E−05 | 1.673 |
| 75 | 0.901 | 1.079 | 0.990 | 11.156 | 3.25E+08 | 487994.4 | 2.29E−05 | 3.104 |
| 171 | 1.040 | 0.944 | 0.992 | 11.182 | 3.79E+08 | 568615.4 | 1.97E−05 | 2.670 |
| 92 | 0.718 | 0.742 | 0.730 | 7.756 | 1.22E+08 | 182264.8 | 4.26E−05 | 5.777 |
| 128 | 1.060 | 1.137 | 1.099 | 12.576 | 3.43E+08 | 515152.4 | 2.44E−05 | 3.315 |
| 111 | 1.035 | 1.105 | 1.070 | 12.203 | 1.01E+08 | 150987.2 | 8.08E−05 | 10.973 |
| 104 | 0.262 | 0.224 | 0.243 | 1.385 | 1.19E+08 | 179094.5 | 7.73E−06 | 1.050 |
| 134 | 0.796 | 0.776 | 0.786 | 8.491 | 7.67E+08 | 1150828.7 | 7.38E−06 | 1.002 |

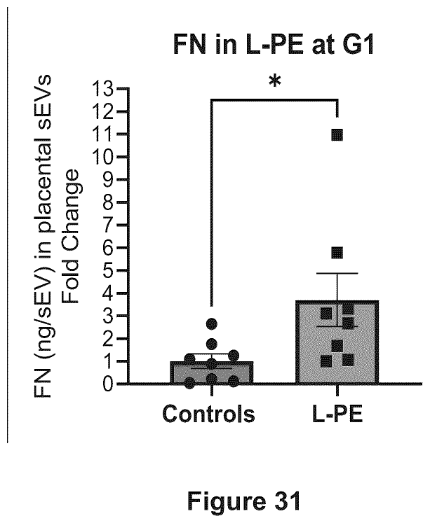

FIG. 31 is a graph showing the fibronectin fold change in placental sEVs, normalized for sEV number.

### Example 3

Maternal endothelial dysfunction and damage are defining features of preeclampsia. Primary microvascular endothelial cells (pMVECs) were obtained from maternal adipose tissue of normotensive term control (TC) and E-PE pregnancies. Characterization of fibronectin levels in control and E-PE pMVECs revealed a striking reduction in fibronectin in E-PE pMVECs. This was accompanied by a substantially reduced angiogenic capacity in E-PE pMVECs, whereby the number and length of tubes and branches formed on a basement membrane were markedly reduced.

Moreover, it was found that exposure of control pMVECs (isolated from normotensive pregnancies) to circulating placental sEVs from E-PE (compared to sEVs from term control) pregnancies significantly decreased fibronectin levels of the pMVECs and stunted their angiogenesis. In addition, treatment of control pMVECs with E-PE sEVs impaired fibril-like fibronectin deposition and diminished its association to the fibronectin receptor, integrin a5β1. Overall, these findings suggest that preeclampsia-induced changes in bioactive fibronectin cargo of circulating placental sEVs affects the homeostasis in distal maternal target cells, such as endothelial cells.

What is claimed is:

1. A method of treating preeclampsia in a subject, the method comprising:
isolating placenta-derived small extracellular vesicles from a blood sample obtained from the subject;
measuring an amount of fibronectin in the placenta-derived small extracellular vesicles;
comparing the amount of fibronectin to a control sample; and
if the amount of fibronectin deviates statistically from the control sample, monitoring the subject for symptoms of preeclampsia.

2. The method of claim 1 further comprising measuring an amount of jumonji C domain containing protein 6 (JMJD6) and comparing the amount of JMJD6 to the control sample.

3. The method of claim 2 wherein a decrease in the amount of JMJD6 and an increase in the amount of fibronectin relative to the control sample predicts preeclampsia.

4. The method of claim 1 further comprising treating the subject with at least one of: hinokitiol, acriflavine, an antihypertensive drug, an anticonvulsant medication, a corticosteroid, an intravenous salt solution, a Caesarean section, induced delivery, and bed rest.

5. The method of claim 1 wherein isolating the placenta-derived small extracellular vesicles comprises:
centrifuging the blood sample to obtain an isolate of small extracellular vesicles; and
filtering the isolate to obtain the placenta-derived small extracellular vesicles.

6. The method of claim 5 wherein isolating the placenta-derived small extracellular vesicles further comprises immunoprecipitating the blood sample using a biotinylated anti-human placental alkaline phosphatase (PLAP) antibody.

7. The method of claim 1 wherein isolating the placenta-derived small extracellular vesicles comprises sorting vesicles in the blood sample using a flow cytometer to obtain the placenta-derived small extracellular vesicles.

8. The method of claim 2 wherein measuring the amount of JMJD6 and measuring the amount of fibronectin comprise conducting an enzyme-linked immunosorbent assay (ELISA) on the placenta-derived small extracellular vesicles.

9. The method of claim 1 wherein the blood sample is obtained from the subject between 10 and 34 weeks of gestation, and the method is for predicting early-onset preeclampsia.

10. The method of claim 1 wherein the blood sample is obtained from the subject after 34 weeks of gestation, and the method is for predicting late-onset preeclampsia.

11. A kit for predicting preeclampsia, the kit comprising:
a biotinylated anti-human placental alkaline phosphatase (PLAP) antibody for isolating placenta-derived small extracellular vesicles from a blood sample; and
an anti-human fibronectin coating antibody for use in an enzyme-linked immunosorbent assay to measure an amount of fibronectin in the isolate of small extracellular vesicles.

12. The kit of claim 11 further comprising an anti-human JMJD6 antibody for use in an enzyme-linked immunosorbent assay to measure an amount of JMJD6 in the isolate of small extracellular vesicles.

13. A kit for predicting preeclampsia, the kit comprising:
a biotinylated anti-human placental alkaline phosphatase (PLAP) antibody for isolating placenta-derived small extracellular vesicles from a blood sample; and
an anti-human fibronectin coating antibody for use in an enzyme-linked immunosorbent assay to measure an amount of fibronectin in the isolate of small extracellular vesicles;
wherein the kit is used according to the method of claim 1.
